(19) United States
(12) Patent Application Publication
(10) Pub. No.: US 2008/0206183 A1
(43) Pub. Date: Aug. 28, 2008

(54) Method of Preparing Grafted Polylysine Dendrimers

(75) Inventors: Auguste Commeyras, Clapiers (FR); Helene Collet, Montpellier (FR); Eddy Souaid, Jounieh (LB); Herve Cottet, Montpellier (FR); Bernard Romestand, Saint Gely Du Fesc (FR); Odile Yvonne Marie Trambouze, Saint Andre De Sangonis (FR)

Correspondence Address: YOUNG & THOMPSON, 209 Madison Street, Suite 500, ALEXANDRIA, VA, 22314, US

(73) Assignee: Central national De La Recherche Scientifique, Paris Cedex 16 (FR)

(21) Appl. No.: 11/912,918

(22) PCT Filed: Apr. 27, 2006
(86) PCT No.: PCT/FR06/00952

§ 371 Date: Mar. 10, 2008

(30) Foreign Application Priority Data
Apr. 28, 2005 (FR) 05/04309

(51) Int. Cl. A61K 31/785; C07K 1/00; A61P 31/10; A61P 31/04; C07K 1/107

(52) U.S. Cl. 424/78.17; 530/333; 527/200

(57) ABSTRACT

The use of monomers of active a-amino acids for the preparation of hydrophobic polypeptides in the form of precipitates, whereby the polypeptides result from the polymerization of the aforementioned monomers of active a-amino acids in an aqueous solvent and can be resolubilized in the solvent.

The present invention relates to a method for the preparation of grafted polylysine dendrimers, the grafted polylysine dendrimers obtained by the implementation of said method, and the use of said polylysine dendrimers, in particular within the framework of the preparation of pharmaceutical compositions.

Two methods are described in the literature for synthesis of the highly branched polylysines. The stepwise method initiated by Denkewalter et al. [1,2] and the NCA aminated acid polymerization method initiated by Klok et al. [3,4].

The first highly branched polylysines (which were classified as regular dendrimers) were prepared as early as 1981 by Denkewalter via successive coupling and deprotection reactions from N^{α},N′^{ε}-di-(ter-butoxycarbonyl)-L-lysine [1,2].

These syntheses consist of adding successive lysine monolayers to a lysine the carboxyl group of which is protected by benzhydrilamine (BHA), to produce progressively the following compounds: BHALys, BHALysLys2, BHALysLys2Lys4, BHALysLys2Lys4Lys8, BHALysLys2Lys4Lys8Lys16, BHALysLys2Lys4Lys8Lys16Lys32, etc.

The coupling reactions are carried out in an anhydrous solvent (CH₂Cl₂, or DMF).

The compounds obtained carry numerous amine functions on the surface. In water they are polycationic.

This method of stepwise synthesis is consistently repeated in the literature.

It was used for example in the work of Matthews et al. [5.6], Baigude et al. [7] and Menz and Chapman [8].

For example, in a first phase Matthews et al. synthesized polylysines identical to those of Denkewalter. In a second phase they modified their surfaces by binding covalently to each free amine function, groups carrying anionic functions (—CO₂⁻, SO₃⁻, etc.) and obtained new polyanionic materials with a polylysine core. These materials were found to have important antiviral [5], antibacterial and antiparasitic [6] properties. Baigude et al. modified the surfaces of the dendrimers by binding their peripheral amine functions to oligosaccharides and used the materials obtained to produce vaccines [7]. Takuro et al. used them in a nascent stage to transfect genes [9]. Choi et al. [10] constructed poly-L-lysine dendrimers stepwise from the two —NH₂s of polyethyleneglycol-α,ω-diamine, and showed that the materials obtained are excellent gene transfectants. These multiple properties appear to underline the benefit of highly branched polylysines as all or part of a large family of novel materials.

These polylysine dendrimers [1.2] are considered to have a maximum branching rate.

Moreover, the benefit of these materials must nevertheless not mask the complexity of the stepwise synthesis method. For example, in addition to the deprotection and purification steps, six steps are necessary for oligocondensation of 63 Lysine units. It is important also to note the need to use non-lysine primers (BHA) in these anhydrous solvents, which can raise questions concerning their possible biological interactions.

Unlike the preceding stepwise synthesis, the second method recently proposed by Klok et al. [3.4] consists of polymerizing the lysine N-carboxyanhydrides in a multi-step procedure for synthesizing highly branched polylysines. Amino acid N-carboxyanhydrides (NCA) are well known peptide precursors [11]. These compounds are sensitive to hydrolysis, [12,13], which does not prevent water being used as a solvent for carrying out certain specific studies [14-17]. Generally, it is nevertheless recommended to polymerize the N-carboxyanhydrides of α-amino acids in an anhydrous medium [11,18,19]. DMF, dioxane and DMSO are among the anhydrous solvents recommended, with a preference for DMF [20].

The authors of this second approach to the synthesis of highly branched polylysines from lysine N-carboxyanhydrides [3] followed the recommendations for use of an anhydrous reaction medium. A method of synthesis in anhydrous DMF resulted from this.

In this method the first step consists of copolymerizing two N-carboxyanhydride lysines differently protected on their epsilon function (Z-Lys-NCA and Boc-Lys-NCA in ratios of 5/1 to 2/1). The protective groups are chosen so that their deprotection can be selective. To avoid losing NCAs by hydrolysis, copolymerization is carried out in a solvent (DMF) which is carefully made anhydrous by the appropriate treatments. Under these conditions, for polymerization to take place, traces of acid resulting from the synthesis of the NCAs must be carefully removed [21]. In anhydrous DMF, the polymerization is primed by an amine (hexylamine for example). The reaction time is 5 days. At the end of the reaction the reaction medium is poured into water, and a linear polylysine poly([Z-L-lysine]-co-[Boc-L-lysine]) is precipitated. After filtration and drying, the product was subjected to an acid treatment (CF₃CO₂H for 1 hour) which suppresses the Boc function, but retains the Z function. Poly([Z-L-lysine]-co-[L-lysine]) is thus formed. It is then washed and carefully dried. This linear poly([Z-L-lysine]-co-[L-lysine]), constitutes a material which the authors call the “core”, from which branched polylysines are obtained in subsequent steps.

This (core) material is made to react, still in anhydrous DMF, for 5 days with a mixture of the two previous NCAs (Z-Lys-NCA and Boc-Lys-NCA) suitably purified. During this reaction, the free amine functions (ex Boc) of this linear polylysine react on the Z-Lys-NCA and Boc-Lys-NCA, and a new {branched poly([Z-L-lysine]-co-[Boc-L-lysine])} is formed. After recovery of the material formed by a method similar to the previous one, this material is subjected to an acid treatment (CF₃CO₂H) which eliminates the Boc functions and retains the Z functions. The product obtained, {branched poly([Z-L-lysine]-co-[L-lysine]) called G0}, is again carefully purified and dried.

This product is then used as a primer in a new reaction in anhydrous DMF for 5 days on a mixture of the two reagents Z-Lys-NCA and Boc-Lys-NCA. After recovery, drying, acid treatment and drying again, a product {branched poly([Z-L-lysine]-co-[L-lysine]) called G1} is obtained.

This product G1, is used in turn as a primer in a reaction in anhydrous DMF for 5 days on a mixture of the two reagents Z-Lys-NCA and Boc-Lys-NCA. After recovery, drying, acid treatment, and drying again, a product {branched poly([Z-L-lysine]-co-[L-lysine]) called G2} is obtained.

The products G0, G1, or G2, are then subjected to a treatment in a very acid medium (HBr/CH₃CO₂H). This treatment suppresses the remaining Z functions and {branched poly(L-lysines) G0, G1, or G2} are obtained.

These {branched poly-L-Lysines} are then precipitated with diethylether and lyophylized.

This method therefore effectively makes it possible to obtain {branched poly-L-lysines} with different structures from those described initially by Denkewalter et al.

These {branched Poly-L-Lysines}[3], are characterized (Table 1), by a grafting rate equal to, or less than 30% deduced from the ratio Hb/Ha≦0.08 of two ¹H-NMR signals. Hb corresponds to the chiral proton of the lysine units bonded at the epsilon position to the neighbouring lysines, Ha corresponds to the chiral proton of the lysine units bonded at the alpha position to the neighbouring lysines, however excluding the protons of the N-terminal lysines (Hc) and C-terminal lysines (Hd) which have a different resonance field. These poly-L-lysines therefore form a network of branched polypeptides (grafting rate less than or equal to 30%) with a primary amine (hexylamine) as polymerization primer.

TABLE 1
Features of the branched polylysines described by Klok et al. [3].

| poly-L-lysines | Mn (g/mole) | | Branching points | |
|---|---|---|---|---|
| of Klok et al. | determined by | | determined by the ratio | Grafting rate |
| [3] | NMR | DPn | 1 H NMR (Hb/Ha) | in % |
| Core | 2 670 | 20 | (Core of the materials of Klok et al) | |
| | | | Linear polylysine of 20 units (Mn = 20) | |
| | | | 6 | 6/20 = 30% |
| | | | determined from the ratio | |
| | | | Hb/Ha = 0.07 of G0 | |
| G0 | 12 400 | 96 | 14 | 14/96 = 14% |
| | | | determined from the ratio | |
| | | | Hb/Ha = 0.08 of G1 | |
| G1 | 30 200 | 235 | 25 | 25/235 = 10% |
| | | | determined from the ratio | |
| | | | Hb/Ha = 0.07 of G2 | |
| G2 | 44 600 | 347 | / | / |

The branching points are the ε-NH₂ functions which have reacted on the NCAs. The grafting rate represents the percentage of the ε-NH₂s which have reacted in relation to the total degree of polymerization of a product of generation n (DPn).

As a result, a subject of the invention is to provide a method for the preparation of polypeptides and in particular polylysine dendrimers, making it possible to reduce the complexity of the methods described above.

In particular, the subject of the present invention is to provide a method for the preparation of polypeptides, and in particular polylysine dendrimers, which is quicker and comprises fewer synthesis and/or purification steps than the known preparation methods.

Another subject of the invention also relates to the dendrimers which can be obtained by implementation of this method.

Finally, another subject of the invention also relates to the use of the dendrimers obtained by implementation of the method of the invention for the preparation of pharmaceutical or antiseptic compositions, as well as analyses of the immunological type.

The present invention relates to the use of activated α-amino acid monomers for the preparation of hydrophobic polypeptides in the form of precipitates, said polypeptides resulting from the polymerization of said activated α-amino acid monomers in an aqueous solvent and capable of being resolubilized in said solvent.

The term “activated α-amino acid monomers” denotes α-amino acids which have undergone a modification that has made them polymerizable under normal temperature and pressure conditions.

The term “hydrophobic polypeptides” denotes polypeptides which are insoluble in water or in the aquo-organic solvents used. The hydrophobic character of a solute (polypeptide) can be estimated by the partition coefficient P of the solute in water and an organic solvent (generally octanol used as a reference). The partition coefficient P is defined as the ratio of the concentration of the solute in the organic phase divided by the concentration in the aqueous phase. The logarithm of P can be estimated by the Rekker constants [22] or determined experimentally by the ratio of the concentrations in the organic phase and in the aqueous phase.

The term “aqueous solvent” denotes any solvent or mixture of miscible solvents the larger part of which comprises water (at least 50% by weight).

The term “resolubilizes” denotes the return of a hydrophobic peptide to an aqueous solution by modification of its Rekker constants, which can be obtained for example by releasing the group protecting the epsilon function of a lysine.

The advantages of this use are that via an elementary cyclical method which is simple to carry out: it is possible 1/ to synthesise peptides of a controlled size and to recover them easily, 2/ to resolubilize them by deprotection, 3/ to increase their size, also in a controlled manner and to recover them equally easily from the reaction medium.

In a particular embodiment of the use defined above, the activated α-amino acids are chosen from α-amino acid N-carboxyanhydrides (NCA), α-amino acid N,N′-carbonyldiimidazoles, carbonyl sulphide α-amino acids, carbonic anhydride α-amino acids, and amino thioacids-oxidizing agents.

The α-amino acid N-carboxyanhydrides (NCA) are prepared according to one of the methods described [23-29] but also by the indirect methods analysed by Pascal et al. [30].

In another particular embodiment of the use defined above, the activated α-amino acids are α-amino acid N-carboxyanhydrides (NCA) of the following Formula (I):

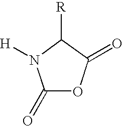

in which R represents a side chain of a natural or modified α-amino acid.

The term natural amino acid denotes the amino acids which are found in living systems.

The term modified amino acid denotes natural amino acids which have undergone a modification on their side chain (R).

In a more particular embodiment of the use defined above, L-lysine-NCA monomers are used for the preparation of polylysine in an aqueous solvent.

The term polylysine denotes any polymer the majority single component of which is lysine.

In L-lysine-NCA monomers, the ε amine group can be protected or not protected.

The term “protection of the amine group” denotes the reversible fixing of a chemical group, called the protective group, onto the amine group so as to reduce or even remove its reactivity and to modify the hydrophobic character of the molecule.

The present invention also relates to a method for the preparation of a grafted homo or heteropolylysine dendrimer, from a primer comprising at least one primary or secondary amine group, comprising a step of addition of an L-lysine-NCA monomer, and optionally one or more other α-amino-acid-NCA monomers, in particular chosen from the list comprising L-ornithine-NCA, L-glutamic-acid-NCA and its γ-amide, L-aspartic-acid-NCA and its β-amide, L-diamino-2.4,-butyric-acid-NCA and its β-amide, L-tyrosine-NCA, L-serine-NCA, L-threonine-NCA, L-phenylalanine-NCA, L-valine-NCA, L-leucine-NCA, L-isoleucine-NCA, L-alanine-NCA, and glycine-NCA, to said primer in an aqueous solvent. These other (NCA) monomers can be used in the last step of construction of the grafted dendrimer to modify the surface functions of the final material.

The term “homopolylysine” denotes a polylysine constituted only of lysine.

The term “heteropolylysine” denotes a polylysine made up of more than 30 mol % lysine and containing also units of a different nature.

The term grafted dendrimer, in particular a grafted polylysine dendrimer, denotes a macromolecule the structure of which is more flexible, more extended and less regular than that of the dendrimers (described initially by Denkewalter [1]) but more structured than that of the branched molecules of Klok et al. [3,4]. Such flexible structures have unique properties which differ from those of the conventional dendrimers, as shown on page 18 of the study “Dendrimers and other dendritic polymers” edited by Frechet and Tomalia [31].

The term “primer” denotes any chemical species capable of initiating a polymerization process. In the case of activated amino acids, primers are nucleophilic species such as: a primary C₁-C₁₂ alkylamine, a C₁-C₁₂ alkyldiamine, a secondary or tertiary C₁-C₁₂ alkylamine, a polyaromatic aryl amine detectable in UV or by fluorescence, a primary or secondary alkyl- or aryl amine carrying a detectable radioactive atom, an amino acid having one or more free amine functions, a peptide having one or more free amine functions, a dendrimer having one or more free amine functions, a polymer having one or more free amine functions.

For synthesis of the dendrimer of generation i (i≧2), the primer used corresponds to the deprotected grafted dendrimer of generation i−1.

During the synthesis of the dendrimer of generation 1 according to the invention, it is possible not to add primer, i.e. an external primer, to the reaction mixture when it is desired to produce peptides comprising only units identical to that of the starting activated amino acid or amino acids (NCA). In this case, hydrolysis of the NCA or NCAs (initiated by the presence of water in the solvent) releases the amino acid(s) required for priming. Thus, for synthesis of the first generation (called P1), an external primer may or may not be used, and in the case where one is used, it may be different from the amino acid monomer used.

In the particular case of the synthesis of homopolylysine dendrimer, if an external primer is not used in the step of synthesis of the first generation product (P1), an identical result is obtained to that obtained by using the ε-protected lysine as a primer. With an unprotected lysine as an external primer, the extension of the chain has as a starting point the two α and ε amine functions. In these two cases the yield of the reaction is 5% greater than in the case where no external primer is used. On the other hand, in each subsequent step leading to the generation 2 (P2), generation 3 (P3) or generation n (Pn) products, the primer used is the product obtained in the step Pn−1.

In a particular embodiment of the method of preparation defined above, the monomer was only L-lysine-NCA.

In another particular embodiment of the method of preparation defined above, the L-lysine-NCA was N^{ε}-protected, in particular by a group chosen from —COH (Formyl), —COCF₃ (TFA), —OCOC(CH₃)₃ (Boc), —COOCH₂Φ (Z),

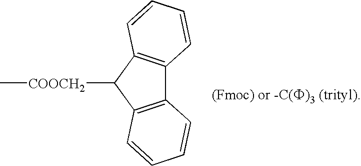

The above groups were classified in increasing order of hydrophobia, their respective Rekker constants are given by log P=−1.528, −0.946, 0.964, 1.124, 2.849, 5.81.

Advantageously, the nature of the protective group allows the size of the polylysine obtained to be modulated. In fact, the more hydrophobic the protective group, the smaller the number of protected lysine units present in the polylysine required to drive precipitation of the polylysine. Thus, the size of the polylysine obtained with weakly hydrophobic protective groups (Formyl) is greater than the size of polylysines obtained with more hydrophobic groups (TFA).

In another particular embodiment of the method of preparation defined above, the primer was chosen from L-lysine, L-ornithine, a homopolylysine, a poly(ethylene glycol)-α,ω-diamine, a heteropolylysine, a heteropeptide, or a homopeptide.

The term homopeptide denotes a peptide all the residues of which are identical.

The term heteropeptide denotes a peptide constituted by residues of a different nature.

In another embodiment of the method of preparation defined above, the pH of the solvent is approximately 3 to 9.

The use of this pH range corresponds to the domain of stability of the peptide bond [32] and leads to an increase in the yield of the reaction relative to the pHs outside this range.

According to a preferred embodiment, the invention relates to a method for the preparation of a generation 1 grafted homo or heteropolylysine dendrimer (P1) as defined above, comprising the following steps:
the addition of the N^{ε}-protected L-lysine-NCA to a primer in a aqueous solvent, at an appropriate pH, in order to obtain a protected generation 1 grafted polylysine dendrimer in the form of a precipitate,
deprotection of the protected generation 1 grafted polylysine dendrimer obtained in the previous step, in order to obtain a generation 1 grafted polylysine dendrimer.

The term “generation” denotes the product obtained during each phase of growth of this macromolecule.

The term “generation 1” denotes the product of the first polymerization reaction of the TFA-L-Lysine NCA, without external primer, or with TFA-L-lysine or any other primer defined above.

It will be noted that according to the invention the grafted dendrimer of generation 1 (P1) is linear.

In a preferred embodiment of the method for the preparation of a generation 1 grafted homopolylysine dendrimer as defined above, the primer is N^{ε}-protected or unprotected L-lysine.

The use of N^{ε}-protected L-lysine as primer allows a homopolypeptide of L-lysine to be obtained.

The use of unprotected L-lysine allows the elongation of two homopolypeptide L-lysine chains starting from α and ε amines respectively.

In another embodiment of the method for the preparation of a heteropolylysine dendrimer of generation 1 as defined above, the primer is a poly(ethylene glycol)-α,ω-diamine, the molecular weight of which can be comprised between 100 Da and 10,000 Da, in particular between 1,000 Da and 10,000 Da.

In another preferred embodiment of the invention, the method for the preparation of a homopolylysine dendrimer of generation 1 as defined above, comprises:
a step of addition of L-lysine-NCA N^{ε}-protected by a TFA group, to an aqueous solution with a pH of approximately 6 to approximately 8, without addition of primer or with an L-lysine primer N^{ε}-protected by a TFA group, in order to obtain a precipitate of protected polylysine dendrimer of generation 1, and
a step of deprotection of the polylysine polymer obtained in the previous step in order to obtain a polylysine dendrimer of generation 1 which was linear, of approximate molecular weight 1,400 Da, in particular 1,450 Da, having a polymolecularity index of approximately 1.2 and corresponding to an average degree of polymerization of 8 units of lysine.

In the above and hereafter, the term “average molecular weight” denotes the number average molecular weight

$${M}_{n} = \frac{{\sum }_{i} {N}_{i} {M}_{i}}{{\sum }_{i} {N}_{i}}$$

where the sum relates to all the possible molecular weights, Nᵢ being the number of molecules having the molecular weight Mᵢ. The weight average molecular weight is defined by

$${M}_{w} = \frac{{\sum }_{i} {N}_{i} {M}_{i}^{2}}{{\sum }_{i} {N}_{i} {M}_{i}} .$$

The polydispersity index (or polymolecularity) is defined by I=M_{w}/Mₙ. Similarly, the number average degree of polymerization is defined by

$${DP}_{n} = \frac{{\sum }_{i} {N}_{i} {DP}_{i}}{{\sum }_{i} {N}_{i}}$$

and the weight average degree of polymerization by

$${DP}_{w} = \frac{{\sum }_{i} {N}_{i} {DP}_{i}^{2}}{{\sum }_{i} {N}_{i} {DP}_{i}} .$$

The term “average degree of polymerization” denotes the average number of monomers contained in the polymer chain.

The (number or weight) average molecular weights, the average degree of polymerization as well as the polymolecularity index were determined absolutely by steric exclusion chromatography coupled with light diffusion and a differential refraction index detector. For the first generation (P₁, low molecular weight polymer) determination of the absolute molecular weight was carried out by capillary electrophoresis. The result obtained was consistent with the results obtained in NMR, and by MALDI-TOF mass spectrometry.

The present invention also relates to a method for the preparation of a grafted homo or heteropolylysine dendrimer of generation n as defined above, in which n is an integer from 2 to 10:
comprising a step of addition of N^{ε}-protected L-lysine-NCA to a primer constituted by a grafted polylysine dendrimer of generation n−1, in an aqueous solvent, at an appropriate pH, in order to obtain the protected grafted polylysine dendrimer of generation n in the form of a precipitate,
   said grafted polylysine dendrimer of generation n−1 being itself obtained from the addition of N^{ε}-protected L-lysine-NCA to a primer constituted by a grafted polylysine dendrimer of generation n−2, in an aqueous solvent, at an appropriate pH, in order to obtain a protected grafted polylysine dendrimer of generation n−1, and the deprotection of said polylysine,
   said grafted polylysine dendrimer of generation n−2 being itself obtained as indicated in relation to the grafted polylysine dendrimer of generation n−1,
   and when n=2, the generation 1 grafted polylysine dendrimer is as defined above, said generation 1 grafted polylysine dendrimer forming the core of the grafted polylysine dendrimer of generation n,
a step of deprotection of the protected grafted polylysine dendrimer of generation n to obtain the grafted polylysine dendrimer of generation n.

The term “appropriate pH” denotes a pH comprised between 3 and 9 and more particularly a pH of 6.5.

In a particular embodiment, the invention relates to a method for the preparation of a grafted homopolylysine dendrimer of generation n (Pn) as defined above, in which the L-lysine-NCA is N^{ε}-protected by TFA, the core of said grafted homopolylysine dendrimer of generation n being formed from a linear polylysine comprising approximately 8 residues of L-lysine, such as can be obtained by the implementation of the method for the preparation of a homopolylysine dendrimer of generation 1 as defined above, and the degree of branching of said grafted homopolylysine dendrimer of generation n being approximately 40% to approximately 100%, in particular approximately 60% to approximately 100%.

The terms “degree of branching”, “grafting rate”, and “branching rate” of a grafted polylysine dendrimer of generation n (Pn), interchangeably denote the percentage of the ε NH₂s of Pn which have reacted in relation to the total degree of polymerization of the product of generation n (DPn).

The preferred means for determining the degree of branching was measurement of the ratio between the intensity of the NMR signals of the Hb and Ha protons. These signals correspond: Hb (4.01 ppm) to the resonance of the proton carried by the chiral carbons of the lysine units bound to the ε amine functions, Ha (4.08 ppm) to the resonance of the protons carried by the chiral carbons of the lysine units bound to the α amine functions excluding the protons carried by the N-terminal (Hc) and C-terminal (Hd) lysine units which resonate between 3.5 and 3.9 ppm. The ratio between the intensity of these two signals makes it possible to ascertain the degree of branching of these grafted dendrimers [3].

Advantageously, the Hb/Ha ratios of the grafted polylysine dendrimers according to the invention are approximately 0.2 to approximately 0.8, in particular approximately 0.25 to approximately 0.60.

In a preferred embodiment, the invention relates to a method for the preparation of a grafted homo or heteropolylysine dendrimer of generation 2 (P2) as defined above, comprising:
a step of addition of the L-lysine-NCA Nε protected by TFA to a primer constituted by a polylysine dendrimer of generation 1,
   said polylysine dendrimer of generation 1 being such as obtained by implementation of the method for the preparation of a homopolylysine dendrimer of generation 1 comprising approximately 8 units of lysine, as defined above,
   the mass ratio (L-lysine-NCA Nε protected by TFA)/(polylysine dendrimer of generation 1) being approximately 2.6 to approximately 3.9, in particular approximately 3,

said step of addition taking place in an aqueous solvent, at an appropriate pH, in order to obtain a protected polylysine dendrimer of generation 2,
a step of deprotection of the polylysine obtained in the previous step, in order to obtain a generation 2 grafted polylysine dendrimer (P2) having an average molecular weight of approximately 6,000 to approximately 14,000 Da, in particular approximately 8,350 Da, preferably approximately 8,600 Da, a polydispersity of approximately 1.4, and approximately 40 to approximately 60, in particular approximately 48, free external —NH₂ groups.

The generation 2 grafted homopolylysine dendrimer above is also characterized in that its Hb/Ha ratio is approximately 0.1 to approximately 0.8, in particular approximately 0.2 to approximately 0.3, more particularly approximately 0.25. Moreover, during the synthesis of the generation 2 grafted homopolylysine dendrimer above, the P1 primer undergoes a grafting rate of approximately 50% to approximately 100%, in particular approximately 80% to approximately 100%, more particularly approximately 100%.

The term “free external —NH₂ groups” denotes the —NH₂ groups of the deprotected ε amine functions and the NH₂ groups in the α position.

The preferred method for determining the presence of the free —NH₂ groups is NMR. For example when N^{ε}-trifluoacetyllysine is used, the removal of the protective group and the emergence of the free amine function are followed in ¹⁹F NMR by the disappearance of the trifluoroacetyl group signal (at −76 ppm) and the simultaneous appearance of the trifluoroacetate signal (at −75.8 ppm). This disappearance is total in 15 hours at 40° C. in a 1 M water/methanol/ammonia solution).

In another preferred embodiment, the invention relates to a method for the preparation of a grafted homopolylysine dendrimer of generation 3 (P3) as defined above, comprising:
a step of addition of the L-lysine-NCA N^{ε} protected by TFA to a primer constituted by a generation 2 grafted polylysine dendrimer,
   said generation 2 grafted polylysine dendrimer being as obtained by implementation of the method defined above,
   the mass ratio (Nε protected L-lysine-NCA by TFA)/(generation 2 grafted polylysine dendrimer) being approximately 2.6 to approximately 3.9, in particular approximately 3,

said step of addition taking place in an aqueous solvent, at an appropriate pH, in order to obtain a generation 3 protected grafted polylysine dendrimer (P3),
a step of deprotection of the polylysine obtained in the previous step, in order to obtain a generation 3 grafted polylysine dendrimer having an average molecular weight of approximately 15,000 to approximately 30,000 Da, in particular approximately 21 500 Da, preferably approximately 22,000 Da, a polydispersity of approximately 1.4, and approximately 100 to approximately 150, in particular approximately 123, free external —NH₂ groups.

The generation 3 grafted homopolylysine dendrimer above is also characterized in that its Hb/Ha ratio is approximately 0.2 to approximately 0.8, in particular approximately 0.5 to approximately 0.7, more particularly approximately 0.6. Moreover, during the synthesis of the generation 3 grafted homopolylysine dendrimer above, the P2 primer undergoes a grafting rate of approximately 50% to approximately 90%, in particular approximately 70% to approximately 90%, more particularly approximately 81%.

In another preferred embodiment, the invention relates to a method for the preparation of a generation 4 grafted homopolylysine dendrimer (P4) as defined above comprising:
a step of addition of the L-lysine-NCA N^{ε}-protected by TFA to a primer constituted by a generation 3 grafted polylysine dendrimer,
   said generation 3 grafted polylysine dendrimer being such as that obtained by the implementation of the method defined above,
   the (L-lysine-NCA Nε-protected by TFA)/(generation 3 grafted polylysine dendrimer) ratio being approximately 2.6 to approximately 3.9, in particular approximately 3,

said step of addition taking place in an aqueous solvent, at an appropriate pH, in order to obtain a protected generation 4 grafted polylysine dendrimer (P4),
a step of deprotection of the polylysine obtained in the previous step in order to obtain a generation 4 grafted polylysine dendrimer having an average molecular weight of approximately 50,000 to approximately 80,000 Da, in particular of approximately 64,000 Da, preferably of approximately 65,000 Da or 65 300 Da, a polydispersity of approximately 1.4, and approximately 300 to approximately 450, in particular approximately 365 free external —NH₂ groups.

The generation 4 grafted homopolylysine dendrimer above is also characterized in that its Hb/Ha ratio is of approximately 0.2 to approximately 0.8, in particular of approximately 0.4 to approximately 0.5, more particularly approximately 0.46. Moreover, during the synthesis of the generation 4 grafted homopolylysine dendrimer above, the P3 primer undergoes a grafting rate of approximately 50% to approximately 90%, in particular of approximately 70% to approximately 90%, more particularly approximately 80%.

In another preferred embodiment, the invention relates to a method for the preparation of a generation 5 grafted homopolylysine dendrimer (P5) as defined above comprising:
a step of addition of the L-lysine-NCA N^{ε}-protected by TFA to a primer constituted by a generation 4 grafted polylysine dendrimer,
   said generation 4 grafted polylysine dendrimer being such as that obtained by the implementation of the method defined above,
   the ratio (L-lysine-NCA Nε protected by TFA)/(generation 4 grafted polylysine dendrimer) being approximately 2.6 to approximately 3.9, in particular approximately 3,

said step of addition taking place in an aqueous solvent, at an appropriate pH, in order to obtain a protected polylysine dendrimer of generation 5,
a step of deprotection of the polylysine obtained in the previous step, in order to obtain a grafted polylysine dendrimer of generation 5 having an average molecular weight of approximately 140,000 to approximately 200,000 Da, in particular approximately 169,000 Da, preferably approximately 172,000 Da or 172 300 Da, a polydispersity of approximately 1.4, and approximately 900 to approximately 1100, in particular approximately 963, free external —NH₂ groups.

The generation 5 grafted homopolylysine dendrimer above is also characterized in that its Hb/Ha ratio is approximately 0.2 to approximately 0.8, in particular approximately 0.3 to approximately 0.5, more particularly approximately 0.4. Moreover, during the synthesis of the generation 5 grafted homopolylysine dendrimer above, the P4 primer undergoes a grafting rate of approximately 50% to approximately 90%, in particular approximately 60% to approximately 70%, more particularly approximately 65%.

The present invention also relates to a method for the preparation of a grafted homo- or hetero polylysine dendrimer as defined above, in which the primer, in particular that used for the preparation of generation 1, is fixed covalently to the grafted dendrimer, said primer comprising a marker product, which makes the dendrimer easily detectable by the following means: UV absorption, fluorescent emission, opacity to X-rays or sensitivity to magnetic fields. The following respective formulae are given as examples of marker products without being limitative: 7-amino-1,3-naphthalene-disulphonic acid, aminofluoresceine, 3-(4-carboxybenzoyl)quinoline-2-carboxaldehyde, rhodamine sulphochloride (Texas Red), polyiodide contrast agents such as amidotrizoates or ioxaglate, or also metal clusters (Au, Fe, Ga, Mn) carrying aldehyde functions such as Au₁₁(P(C₆H₄)CONHCH₂CHO)₃)₇), carboxylic acid functions or other active functions on the amines, used in medicine for diagnostic purposes to reinforce the contrast in imaging under X-ray and by magnetic resonance (MRI). When the above-mentioned marked molecules carry amine functions, such as for example the first two mentioned, they can be used to prime the polymerization of the NCAs. They are then bonded covalently to the first generation grafted dendrimer (P1) by an amide bond on the C terminal. When for example by contrast the marked molecules carry functions such as aldehydes, sulphochlorides, carboxylic acids which are capable of reacting on the amines, then it is possible, by using conventional methods, to bind them covalently to the N terminal of the dendrimer (P1) before deprotection of the N^{ε}-TFA functions. These operations are carried out in DMF, and the reaction product is then recovered by precipitation in water. After releasing the N^{ε}-TFA functions, the construction of the dendrimer is continued. As from the second generation of the dendrimer, and a fortiori the third, the marker products are “buried” in the core of the dendrimer. They are thus invisible to immune systems like the dendrimer itself, as shown in example 22 below. By being carried by the dendrimers, these marker products have become soluble in physiological media. The toxicity of these marker products (when used alone) is masked from the organisms by the dendrimers. These marker products can then be monitored by UV absorption, fluorescence, X-ray or magnetic resonance imaging (MRI) according to the nature of the marker product.

The method for the preparation of a grafted polylysine dendrimer according to the invention has numerous advantages.

A first advantage is that in an aqueous solvent the activated monomer (TFA-L-Lys-NCA) can be used as a crude reaction product, i.e. with no previous purification, contrary to the recommendations of the literature [21]. The hydrochloric or nitrous acids, formed during the synthesis of the NCAs [25, 29, 32] are not, in an aqueous solvent, in any way problematic, whatever the pH comprised between 3 and 9 to which the polymerization is taken.

A second advantage is that the polymerization reactions according to the invention are rapid. They are complete in a time comprised between 2 hours and 30 min, as a function of the temperature comprised between −10 and +60° C., and pH comprised between 3 and 9.

A third advantage is that the polypeptides formed (whatever the generations) precipitate in an aqueous solvent and are easily recoverable by filtration or centrifugation. This advantage is increased by the fact that the peptides which precipitate have a low polydispersity index. In fact, the polymers of short amino acids are soluble, which allows their elongation, but precipitate beyond a certain length, which blocks their elongation. The consequence is that the products obtained have a narrow distribution curve, the average mass of which depends in particular on the hydrophile-hydrophobe balance between the initial carrier (primer) and the final product. This balance is also a function of the nature of the group used for ε-protection of the amine function.

A fourth advantage of the method of the invention is that in an aqueous medium the polymerization reactions can be primed in an acid medium, unlike the previous methods which require anhydrous solvents. Thus, the first polymerization reaction is primed either by TFA-L-Lys originating from hydrolysis of the NCAs, or by TFA-L-Lys added to the reaction medium, such that after release, the polylysine dendrimer of generation 1 (P1) is strictly constituted by lysine. This qualitative (biologically beneficial) purity is necessarily present in the following generations, since P1 is the primer for the generation 2 grafted polylysine dendrimer (P2), which is the primer for the generation 3 grafted polylysine dendrimer (P3) etc.

This possibility does not prevent the polymerization being primed by amines other than the preceding ones, such as for example amino acids, peptides, alkyl amines, alkyldiamines, arylamines.

A fifth advantage of the method of the invention is that it is easy to control, and to programme the number average weights (Mn) of the grafted polylysine dendrimers of the expected generation i (Pi). In the reaction of formation of the grafted polylysine dendrimers, the choice of the ratio {TFA-L-Lys-NCA}/primer, is in fact important. For an ad hoc ratio the grafted polylysine dendrimer obtained is monomodal, having low polydispersity and a given Mn. But if an excess of activated monomer is used, the average Mn of the Pi obtained is higher that when the ad hoc ratio is used. A too high excess of activated monomer leads, besides the expected Pi, to the formation of traces of P1 that are eliminated by microfiltration. This property thus makes it possible to obtain for each generation, grafted polylysine dendrimers of number average weights which are 30% more controllable than the ad hoc average weights. Consequently, by starting from a generation P1 of Mn 1,450 Da, it is possible to obtain grafted polylysine dendrimers of variables weights comprised between the following minima and maxima:

6,000 Da<Mn P2<14,000 Da, 15,000 Da<Mn P3<30,000 Da, 50,000 Da<Mn P4<80,000 Da, 140,000 Da<Mn P5<200,000 Da.

Preferably:

Mn P2<12,000 Da, Mn P3<28,600 Da, Mn P4<84,000 Da, Mn P5<224,000 Da.

By varying the nature of the protective group of the epsilon amine function, it is possible to obtain, depending on the hydrophobia of this group, compounds of P1 to Pi of a lower Mn if the protective group is more hydrophobic, or higher if the protective group is more hydrophilic. This method therefore makes it possible to synthesize grafted polylysine dendrimers having the desired average weights (Mn) very easily.

The grafted polylysine dendrimers of the invention have molar weights which increase more quickly as a function of the generation number than for lysine dendrimers of the prior art, due to the fact that the primer that can be used for generation 2 is a linear polylysine. In this respect, although the grafted polylysine dendrimers of the invention can be compared to the branched polylysines described by Klok et al. [3], they differ structurally from the latter by their much higher grafting rate.

For these reasons, the polylysine dendrimers of the invention are called Grafted Lysine Dendrimers (GLD).

The present invention also relates to a grafted polylysine dendrimer such as can be obtained by the implementation of a method of preparation as defined above, in particular by the implementation of a method for the preparation of a grafted homo or heteropolylysine dendrimer of generation n, where n is greater than 1, as defined above.

In a particular embodiment, the grafted lysine dendrimer (or grafted polylysine dendrimer) as defined above is characterized in that the external —NH₂ groups are totally or partially bonded covalently or non-covalently to the groups chosen from the list comprising monosaccharides, nucleic acids, proteins or groups carrying carboxylic, sulphonic, phosphoric functions, ethylene polyoxides, hydrocarbonated or perfluorohydrocarbonated chains, aldehydes or their precursor, or masked reactive functions (isocyanates) such as carbamoyl or chloroethylnitrosourea groups. These surface function modifications confers on them novel physical properties and, moreover, novel biological properties.

In another particular embodiment, the grafted lysine dendrimers (or grafted polylysine dendrimers) as defined above are characterized in that they can be fixed onto a support covalently or non-covalently, in particular by electrostatic bonds, allowing the support to retain its properties.

A first advantage is that by retaining their properties, said supports (particles, fibres, or surfaces) acquire the properties of the dendrimers. Implementation of these supports then allows them to be used as filters, tissues, surface coatings, abrasives particles, in various environments.

The second advantage is linked to the fact that in fixing the grafted dendrimers of the present invention onto a support, a large number of reactive functions (amines or acids) per surface unit of these supports are created and their properties are thus modified. Advantage is then taken of this modification, for example for fixing onto said supports concentrations of antibodies, aptamers or fragments of DNA greater than those capable of being fixed onto the plates normally used for immunological assays, which allows an increase in the sensitivity of these assays from these highly specific receptors.

A third advantage is that the grafted polylysine dendrimer thus fixed retains its properties in relation to its non-fixed condition.

The term support denotes inorganic materials (silica, alumina, metal oxides) or organic materials (polyacrylics, polyurethanes, polyepoxy, polyisocyanate, polysaccharides, polyamides).

According to a preferred embodiment, the grafted polylysine dendrimers of the invention are furtive vis-à-vis the immune systems with which they are brought into contact, and can advantageously be used as carriers of haptens or of antigens against which immune systems react to form antibodies.

The present invention also relates to the use of the grafted polylysine dendrimers defined above, for the preparation of antigen-grafted polylysine dendrimer complexes or hapten-grafted polylysine dendrimer complexes, intended for the production of antibodies directed against said antigen or said hapten.

In fact, the grafted lysine dendrimers according to the invention are characterized in that they are furtive vis-à-vis the immune systems, i.e. that the immune systems do not produce antibodies directed against the grafted lysine dendrimers. However, if the grafted lysine dendrimers according to the invention carry a hapten or an antigen fixed to their surface, immune systems can produce antibodies against these haptens or antigens without creating any against the grafted lysine dendrimer carrier, in contrast to what takes place when the carriers are proteins such as BSA (bovine saline albumin), HSA (human saline albumin), and KLH (Keyhole Limpet Hemocyanin).

The advantage is that the antibodies thus produced specifically against the antigens and the haptens are not mixed with antibodies specific to the carrier, in contrast to what takes place when they are produced by conventional techniques, and are more directly usable as analytical tools subsequently.

The term hapten denotes a molecule of low molar weight which is non-antigenic by itself.

The present invention also relates to a grafted lysine dendrimer (or grafted polylysine dendrimer) composition such as can be obtained by the implementation of a method of preparation as defined above.

The present invention also relates to a grafted lysine dendrimer (or grafted polylysine dendrimer), as defined above, as an antibacterial or anti-fungal, with the proviso that it not be used for the therapeutic treatment of the human or animal body.

In particular in homogeneous phase, the grafted polylysine dendrimer (or grafted lysine dendrimer) according to the invention is used for the treatment of surfaces within the framework of an antiseptic treatment or to prevent risks of microbiological contamination of these surfaces.

The grafted lysine dendrimers can also be used for the treatment of reservoirs of liquids at risk of microbiological contamination, such as water reserves for example.

The grafted polylysine dendrimers of the invention can be used, fixed onto a support as defined above, as antimicrobial filters and coatings, as circulating antimicrobial particles to fight against biofilms, as anti-biofilm coating, in particular to protect ship hulls against fouling.

The present invention also relates to a pharmaceutical composition characterized in that it contains as active ingredient at least one grafted polylysine dendrimer (or grafted lysine dendrimer) as defined above, in combination with a pharmaceutically acceptable vehicle.

The present invention also relates to the use of a grafted polylysine dendrimer (or grafted lysine dendrimer) as defined above, for the preparation of a medicament intended for the treatment of bacterial or fungal infections or cancers.

In a particular embodiment of the use as defined above of a grafted polylysine dendrimer (or grafted lysine dendrimer) for the preparation of a medicament, the bacterial infections are chosen from the infections by GRAM− and GRAM+ bacteria belonging in particular to the families chosen from the list comprising the Pseudomonadaceae, in particular of the genus Pseudomonas, the Legionellaceae, in particular of the genus Legionella, the Enterobacteriaceae, in particular of the genera Escherichia, Salmonella, Shigella, and Yersinia, the Vibrionaceae, the Pasteurellaceae, the Alcaligenaceae, in particular of the genus Bordetella, the Brucellaceae, in particular the genus Brucella, the Francisellaceae, in particular of the genus Francisella, the Neisseriaceae, the Micrococcaceae, in particular of the genera Staphylococcus, Streptoccus, and Listeria.

### DESCRIPTION OF FIGURES

FIG. 1

Chromatograms obtained for the different generations of polylysine dendrimers of the invention (P1 to P5) in SEC (Steric Exclusion Chromatography) coupled with static light diffusion (SL). Experimental conditions: Superose 12 column (Amersham Pharmacia Biotech); flow rate 0.4 mL/min, temperature: 25° C., eluent 1, injected concentrations: 5 g/L, volume of injection: 100 μL. The chromatograms show the refractive index signal (y-axis) in relation to the elution volume (in mL, x-axis).

FIG. 2A and FIG. 2B

FIGS. 2A and 2B represent the variation in the number average molar weight (in g/mol, y-axis) of the polylysine dendrimers of the invention (diamonds) in relation to the generation (x-axis) and comparison with the theoretical molar weights of the monofunctional dendrimers (squares) and difunctional dendrimers (triangles) described by H. A. Klok et al.

The molar weights of the GLDs were determined by SEC coupled with light diffusion (P₂ to P₅) and by capillary electrophoresis (P₁). The molar weights of the polylysine dendrimers were calculated on the assumption that the dendritic structure is perfect (all the amine functions of the lysine monomers are assumed to react). The core of the monofunctional dendrimer corresponds to benzhydrylamine. The core of the difunctionnel dendrimer corresponds to 1,4-diaminobutane.

The equations obtained by non linear adjustment of the experimental data according to an exponential law are shown in the figures with the correlation coefficients R².

FIG. 3

FIG. 3 represents the hydrodynamic range of the grafted lysine dendrimers of the invention (in nm, y-axis) as a function of the generation (x-axis) determined using three different experimental methods: TDA (Taylor diffusion analysis, squares), SLD (Semi-elastic light diffusions, diamonds), and 3D-SEC (triple detection Steric Exclusion Chromatography, light diffusion, viscosimetric detector and refraction index, triangles).

FIG. 4

FIG. 4 represents the hydrodynamic range of the grafted lysine dendrimers of the invention (in nm, y-axis) as a function of the generation (x-axis) determined by TDA in the three buffers studied in the examples, buffer 1 (diamonds), buffer 2 (squares), buffer 3 (triangles).

FIG. 5

FIG. 5 represents the hydrodynamic range (in nm) of the GLDs as a function of the molar weight (in g/mol), on a bi-logarithmic scale. The straight lines represent the linear adjustments corresponding to the relation Rₕ˜M^{1/v}. The values of v are equal to: 2.72±0.22 (buffer 1) (squares); 2.80±0.09 (buffer 2) (rounds); 2.74±0.08 (buffer 3) (triangles).

FIGS. 6A, 6B, 6C, 6D, 6E, 6F, 6G, 6H and 6I

FIGS. 6A, 6B, 6C and 6D represent the ¹H NMR spectra at 400 MHz in D₂O at 25° C. between 3.7 and 4.4 ppm, of linear tri- (FIG. 6D) and pentalysines (FIG. 6C), as well as those of the grafted lysine dendrimers of the invention (generations 1 to 5 (P1 to P5) (FIGS. 6A and 6B).

FIG. 6H represents the chemical formula of trilysine; FIG. 6G represents the chemical formula of pentalysine; FIG. 6F represents the chemical formula of P1 and FIG. 6E represents the chemical formula of P2. The spectral region shown corresponds to the proton carried by the chiral carbon of the L-lysines. Depending on its environment, this proton has different resonance fields. FIG. 6I represents the 4 different environments (Ha, Hb, He, Hd) observed in these different products. Protons Ha, Hb, Hc and Hd carried by the chiral carbons are respectively represented by a white circle, a black circle, the absence of a circle and a grey circle. Ha corresponds to the chiral proton of a core lysine bound to its neighbour by a peptide bond. Hc corresponds to the chiral proton of an N-terminal lysine. Hd corresponds to the chiral proton of a C-terminal lysine. Hb corresponds to the chiral proton of a core lysine of the dendrimer bonded to the NH₂ side chain of a neighbouring lysine. This bond is of amide and non-peptide type.

In the trilysine spectrum (FIG. 6D), the presence of three different signals of equal intensity, Ha, He and Hd, is noted. In the pentalysine spectrum (FIG. 6C), the intensity of Ha is three times greater (3 core lysines) than that of He or Hd which are of equal intensity. By comparison, in the spectrum of compound P1 (FIG. 6B), the intensity of the signal Ha is approximately 7 times greater than that of the signals He or Hd which are of equal intensity, consequently compound P1 is a linear polylysine of average length equal to 9 lysines (7 core and 2 terminal). The Hb signals characterizing the amide bonds are only observable on the spectra of compounds P2, P3, P4, P5. In each of these spectra, the intensity of the Hb signal is proportional to the number of lysines bonded to the side chains of the neighbouring lysines (εNH₂) by amide bonds. Ha is proportional to the number of lysines bonded to their neighbour in α-NH₂ by a peptide bond (with the exception of the N-terminal and C-terminal residues). The Hb/Ha ratio is thus linked to the number of side chains (εNH₂) which in a given generation have reacted on the NCAs in order to produce the following generation.

The ratio Hb/Ha makes it possible to determine the grafting (or branching) rate of a polylysine of generation (n). This grafting rate is the percentage of the εNH₂s of a generation (n) which have reacted on the NCAs in order to produce the generation (n+1).

This grafting rate (Table 6 is 100% for the passage from P1 to P2 (Hb/Ha=0.25), which means that the 8 side chains of the P1 have reacted on the NCAs in order to produce P2, 81% for the passage from P2 to P3 (Hb/Ha=0.60), which means that out of 48 side chains of the P2, 39 have reacted on the NCAs in order to produce P3, 80% for the passage of P3 to P4 (Hb/Ha=0.46), 65% for the passage from P4 to P5 (Hb/Ha=0.40), and 64% for the passage from P5 to P6 (Hb/Ha=0.42) (not shown in the figure).

FIG. 7

Diagrammatic representation of the grafted lysine dendrimers of generation 1 (P1) to generation 5 (P5). Definition of the Pn structure: P1, a=8 (DPn=8); P2, structure of P1 and

$${\sum }_{i = 1}^{2} {b}_{i} = 40 ( DPn = 48 ) ;$$

P3, structure of P2 and

$${\sum }_{i = 1}^{4} {c}_{i} = 98 ( DPn = 123 ) ;$$

P4, structure of P3 and

$${\sum }_{i = 1}^{8} {d}_{i} = 237 ( DPn = 365 ) ;$$

P5, structure of P4 and

$${\sum }_{i = 1}^{16} {e}_{i} = 614 ( DPn = 963 ) {.}_{ }$$

FIG. 8

FIG. 8 shows the electropherogram demonstrating the separation of deprotected P₁ (linear poly-L-lysine) under the following experimental conditions: Virgin silica capillary, 60 cm (50 cm in the detection window)×50 μm. Electrolyte: phosphate buffer (125 mM H₃PO₄ 125 mM Na⁺H₂PO₄⁻ pH=2.16) with 5% dextran. Tension applied: +20 kV. UV detection at 200 nm. Concentration of the sample: 3 g/l. Injection: 2 psi for 9 seconds. This figure represents the absorbance in mAU as a function of the time in minutes.

This electropherogram compared to that obtained by analysing, under the same conditions, linear di- tri-, tetra- and penta-lysines (commercial peptides obtained from Sigma-Aldrich) confirms that the compound (deprotected P1) is a mixture of linear oligolysines of lengths comprised between 3 and approximately 20 lysine residues. From this electropherogram it is possible to determine the number average degree of polymerization (DPₙ=8) and the polymolecularity index (I=1.2). The number average DPₙ and weight average DP_{w} degrees of polymerization were calculated from the following ratios:

$${DP}_{n} = \frac{{\sum }_{i = 1}^{∞} {iN}_{i}}{{\sum }_{i = 1}^{∞} {N}_{i}} and \qquad {DP}_{w} = \frac{{\sum }_{i = 1}^{∞} {i}^{2} {N}_{i}}{{\sum }_{i = 1}^{∞} {iN}_{i} }$$

where Nᵢ is the number of macromolecules having a degree of polymerization i. In the case of UV detection where the absorption is due to the monomers, the response of the detector is proportional to the mass concentration of the polymer. DPₙ and DP_{w} can then be expressed as a function of the height hᵢ (or, in the case where the peaks can be integrated, as a function of the area corrected by the migration time, Aᵢ) of each electrophoretic peak corresponding to the polymer of degree of polymerization i:

$${DP}_{n} = \frac{{\sum }_{i = 1}^{∞} {h}_{i}}{{\sum }_{i = 1}^{∞} \frac{{h}_{i}}{{DP}_{i}}} = \frac{{\sum }_{i = 1}^{∞} {A}_{i}}{{\sum }_{i = 1}^{∞} \frac{{A}_{i}}{{DP}_{i}}} and \qquad {DP}_{w} = \frac{{\sum }_{i = 1}^{∞} {ih}_{i}}{{\sum }_{i = 1}^{∞} {h}_{i}} = \frac{{\sum }_{i = 1}^{∞} {iA}_{i}}{{\sum }_{i = 1}^{∞} {A}_{i}}$$

FIGS. 9A and 9B:

FIGS. 9A and 9B represent the (MALDI-TOF) mass spectra produced on an Applied Biosystems Voyager OF-STR device in reflectron and linear modes. The length of the flight tube is 2 m (linear) or 3 m (reflectron) and the laser wavelength λ=337 nm (laser N₂). The matrix is α-cyano-4-hydroxycinnamic acid (CHCA) at 10 g·L⁻¹ in DMF for the TFA-ε-protected P1 and in CH₃CN/H₂O for the deprotected P1. The sample is prepared in a proportion of 10/1 (v/v) matrix/sample and subsequently (1 μl) deposited on the target. The spectrum is obtained by accumulation of 300 laser shots and the acceleration voltage set to 20 kV.

FIG. 9A corresponds to TFA-protected P1 washed at 0° C. with pure water and FIG. 9B corresponds to deprotected P1, washed at 0° C. 4 times with a solution of ammonium bicarbonate. These figures represent the percentage intensity of the peaks as a function of the mass m/z.

These perfectly reproducible spectra of TFA-protected P1 and deprotected P1 compounds show that when the polymerization of the N′-TFA-L-Lysine NCA is brought to pH 6.5 in water, without primer, a linear polylysine (P1) is obtained without apparent by-products, and “living”, i.e. with a —CO₂H function on the C-terminal residue and a —NH₂ function on the N-terminal residue.

FIG. 10

FIG. 10 is the MALDI-TOF mass spectrum of the product of the polymerization reaction of N^{ε}-TFA-L-Lysine NCA in water at pH 6.5, in the presence of amino-fluorescein. The precipitate obtained in this reaction is washed 4 times by a solution of HCO₃Na 0.1 N, treated with an ammonia solution, and lyophilized. The mass spectrum of the lyophilizate shows that the product obtained is a fluorescein amide (⁺H₂(Lys)ₙ-NHC₂₀H₁₁O₅) having a lysine number n comprised between 2 and 20. For λ excitation at 490 nm, this material fluoresces in a 0.1 M solution of bisodium carbonate at pH 8.5 with a maximum λ at 517 nm and a detectability threshold of less than 10⁻³ mg·L⁻¹. This max emission λ is to be compared with that of the free aminofluoresceine (513 nm) under the same conditions.

FIG. 11

FIG. 11 represents the average weighted development (in g, y-axis) as a function of the time (in days, x-axis) in a group of male Sprague-Dawley rats (n=6) receiving on the first 4 days 360 mg of grafted lysine dendrimer (GLD) P3 and each following day for 180 days, 30 mg GLD P3 of the invention (black curve). The control rats (n=100) receive 0.5 ml of physiological saline (solvent of the solution) (grey curve).

FIG. 12

FIG. 12 represents the percentage survival (y-axis) in relation to a control (diamonds) of Legionella pneumophila bacteria placed in contact with grafted polylysine dendrimers of the invention of generation 2 at 10 mg/L (circles), generation 2 at 100 mg/L (triangles), generation 3 at 10 mg/L (squares), and generation 3 to 100 mg/L (stars) as a function of time (in days, x-axis).

FIG. 13

ELISA on Maxisorp support plates at different dilutions. LOT A D65 on a support plate coated with GLD-hapten (black circles) or GLD native (white circles), LOT B on a support plate coated with GLD-hapten (black triangles) and LOT C on a support plate coated with native GLD (crosses). The absorbance (OD) is shown as a function of the serum dilution logarithm.

FIG. 14

ELISA Covalink. LOT A D21 (black triangles), LOT A D36 (black squares), LOT B (dashes). The absorbance (OD) is shown as a function of the serum dilution logarithm.

FIG. 15

ELISA on Maxisorp support plates at different saline dilutions for LOT A at D65 on a support plate coated with the BSA-hapten (black squares) or native BSA (crosses). The absorbance (OD) is shown as a function of the serum dilution logarithm.

### EXAMPLES

### Example 1

### Polylysine Dendrimer of Generation 1 (P1)

1/ 94 g of N^{ε}-TFA-L-Lysine-NCA is synthesized by a conventional method, in particular as described or reproduced by Collet et al. [29] for example.

2/ 1 litre of a 0.1 M aqueous solution of HCO₃Na maintained at 0° C. is added to the crude product of the above reaction.

3/ After 30 nm the white precipitate of linear Poly-N^{ε}-TFA-L-Lysine formed in the medium was recovered by filtration or centrifugation.

4/ This precipitate, placed in 1 litre of aqueous ammonia solution at pH 11 for 15 hours at 40° C. loses the TFA protective groups of its side chains and becomes soluble in water.

5/ The volume of the solvent is reduced by half in a rotary evaporator.

6/ After lyophilization 54 g of a linear poly-L-Lysine is obtained, with polydispersity 1.2 (measured by CE (Capillary Electrophoresis, see Example 7). Its absolute average mass, measured by capillary electrophoresis (CE) and confirmed by MALDI-TOF and NMR, is 1,450 Da.

### Example 2

### Grafted Lysine Dendrimer (GLD) of Generation 2 (P2)

1/ 94 g of monomer N^{ε}-TFA-L-Lysine-NCA is synthesized as previously.

2/ 1 litre of a 0.1 M aqueous solution of HCO₃Na maintained at 0° C., in which 30 g of the generation 1 product of Example 1 as polymerization primer is dissolved, is added to the crude product of the reaction.

3/ After 30 mn the white precipitate of grafted Poly-N^{ε}(-TFA-L-Lysine formed in the medium is recovered by filtration or centrifugation.

4/ This precipitate, placed in 1 litre of aqueous ammonia solution at pH 11 for 15 hours at 40° C., loses the TFA protective groups of the side chains of the units added during polymerization and becomes soluble in water.

5/ The volume of the solvent is reduced by half in a rotary evaporator.

6/ After lyophilization, 70 g of grafted poly-L-lysine dendrimer of polydispersity 1.40 (measured in SEC) is obtained. Its average mass measured by SL is 8,600 Da.

By varying the monomer mass/primer ratio from 3.9 to 2.6, generation 2 products are obtained, the weights of which vary between 8 600 Da and 12,000 Da.

### Example 3

### Grafted Lysine Dendrimer (GLD) of Generation 3 (P3)

1/ 94 g of N^{ε}-TFA-L-Lysine-NCA is synthesized as previously.

2/ 1 litre of a 0.1 M aqueous solution of HCO₃Na maintained at 0° C., in which 30 g of the generation 2 product of Example 2 is dissolved, is added to the crude product of the reaction.

3/ After 30 min, the white precipitate of grafted Poly-N′-TFA-L-lysine dendrimer, formed in the medium, is recovered by filtration or centrifugation.

4/ This precipitate, placed in 1 litre of aqueous ammonia solution at pH 11 for 15 hours at 40° C., loses the protective TFA groups of the side chains of the units added during the polymerization and becomes soluble in water.

5/ The volume of the solvent is reduced by half in a rotary evaporator.

6/ After lyophilization, 70 g of a grafted poly-L-Lysine dendrimer of polydispersity 1.46 (measured in CES) is obtained. Its average mass measured by SL, is 22,000 Da.

By varying the monomer mass/primer ratio from 3.9 to 2.6, generation 3 products are obtained, the weights of which vary between 22,000 Da and 28,600 Da.

### Example 4

### Grafted Lysine Dendrimer (GLD) of Generation 4 (P4)

1/ 94 g of N^{ε}-TFA-L-Lysine-NCA is synthesized as previously.

2/ 1 litre of a 0.1 M aqueous solution of HCO₃Na maintained at 0° C., in which 30 g of the generation 3 product of Example 3 is dissolved, is added to the crude product of the reaction.

3/ After 30 min, the white precipitate of Poly-N′-TFA-L-Grafted lysine dendrimer, formed in the medium, is recovered by filtration or centrifugation.

4/ This precipitate, placed in 1 litre of aqueous ammonia solution at pH 11 for 15 hours at 40° C., loses the protective TFA groups of the side chains of the units added during the polymerization and becomes soluble in water.

5/ The volume of the solvent is reduced by half in a rotary evaporator.

6/ After lyophilization, 70 g of a grafted poly-L-Lysine dendrimer of polydispersity 1.36 (measured in SEC) and of average mass 65,300 Da (measured by LS) is obtained.

By varying the monomer mass/primer ratio from 3.9 to 2.6, generation 3 products are obtained, the weights of which vary between 65,300 Da and 85,000 Da.

### Example 5

### Grafted Lysine Dendrimer (GLD) of Generation 5 (P5)

1/ 94 g of N^{ε}-TFA-L-Lysine-NCA is synthesized as previously.

2/ 1 litre of a 0.1 M aqueous solution of HCO₃Na maintained at 0° C., in which 30 g of the generation 4 product of Example 4 is dissolved, is added to the crude product of the reaction.

3/ After 30 min, the white precipitate of grafted Poly-N′-TFA-L-Lysine dendrimer of generation 5, formed in the medium, is recovered by filtration or centrifugation.

4/ This precipitate, placed in 1 litre of aqueous ammonia solution at pH 11 for 15 hours at 40° C., loses the protective TFA groups of the side chains of the units added during the polymerization and becomes soluble in water.

5/ The volume of the solvent is reduced by half in a rotary evaporator.

6/ After lyophilization, 70 g of a grafted Poly-N^{ε}-TFA-L-Lysine dendrimer of polydispersity 1.46 (measured in SEC) is obtained. Its average mass measured by SL is 172 300 Da.

By varying the monomer mass/primer ratio from 3.9 to 2.6, generation 3 products are obtained, the weights of which vary between 172,300 Da and 224,000 Da.

### Example 6

### Grafted Lysine Dendrimers (GLD) of Generations 6, 7 and 8 (P6, P7 et P8)

This synthesis was repeated for the following generations 6, 7, 8. Their weights (Mn) assessed by extrapolation are in order equal to 430,000 Da, 1,130,000 Da, and 3000,000 Da.

### Example 7

### Determination of the Average Molar Weights and the Polydispersity Indices

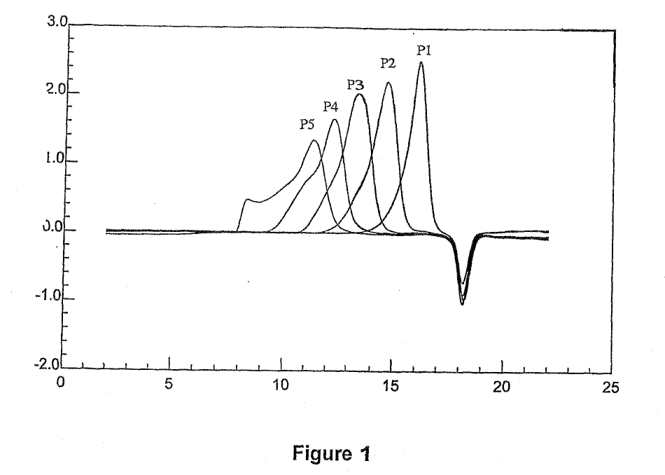

Steric exclusion chromatography coupled with multi-angle static light diffusion and a differential refraction index detector (2D-SEC) made it possible to obtain the molar mass distribution of the 4 GLD generations of (P2-P5). The molar mass distribution of the linear P1 core was obtained by EC (confirmed by MALDI-TOF and NMR). The measurements were carried out in an aqueous eluent constituted by 50 g/L of H₂PO₄⁻Na⁺ (eluent 1, for the definition of the eluents, see Table 2) at pH 4.5. The chromatograms obtained are shown in FIG. 1. Although it was observed that humidity-protected storage of the GLDs made it possible to reduce the formation of aggregates, nevertheless it was not possible to avoid this phenomenon entirely. Thus, in spite of these precautions, FIG. 1 shows the formation of aggregates (shoulders at the low elution volumes), in particular for P4 and P5. The average molecular weight results are shown in Table 3, with the refraction index increment values (dn/dC) and the estimated degrees of polymerization. It should be noted that although this method allows a determination of the absolute molar weights, the molar mass values given must take into account a certain proportion of counter-ions (H₂PO₄⁻ phosphate ions in the eluent). This proportion, although it is difficult to assess experimentally, can be estimated by the theory of condensation described by Manning. Within the framework of this theory, the condensation rate is given by the ratio between the average distance between two charges in the polylysine (0.34 nm) and the Bjerrum length (0.69 nm at 25° C.). The average molar mass of a charged monomer in a pH 4.5 phosphate buffer is therefore: 129×0.49+227×0.51=179 g/mol. From this average, it is possible to estimate the average number of monomers N. The results of molar weights obtained in the buffer 2 by 3D-SEC (Viscotek) show much higher molar mass values, in particular for P4 and P5, probably due to the formation of aggregates in greater quantity. Subsequently, the values obtained by 2D-SEC will be retained.

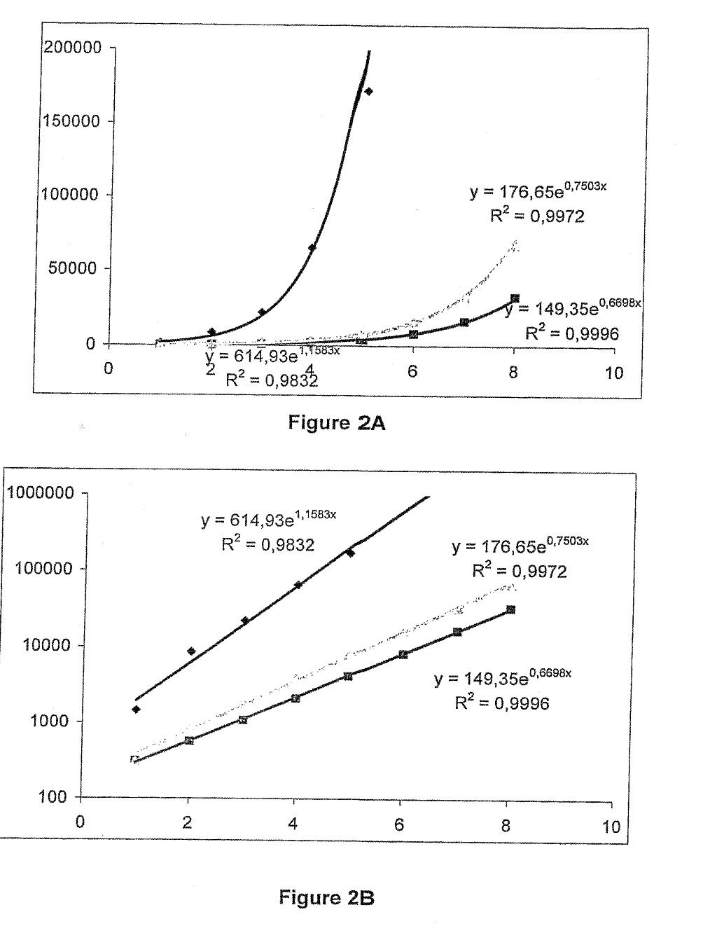

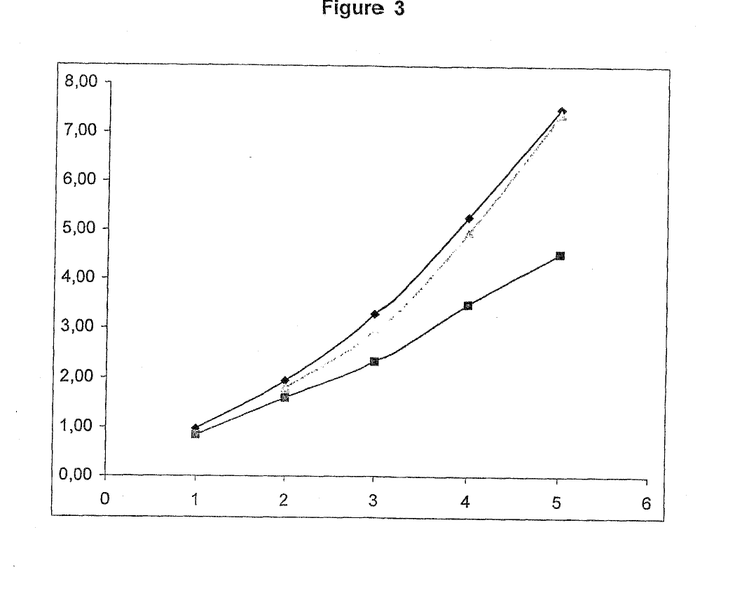

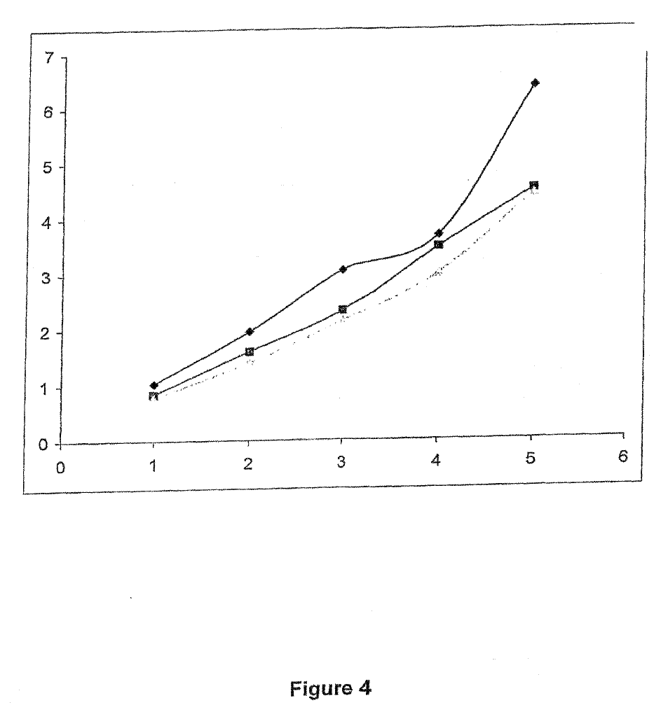

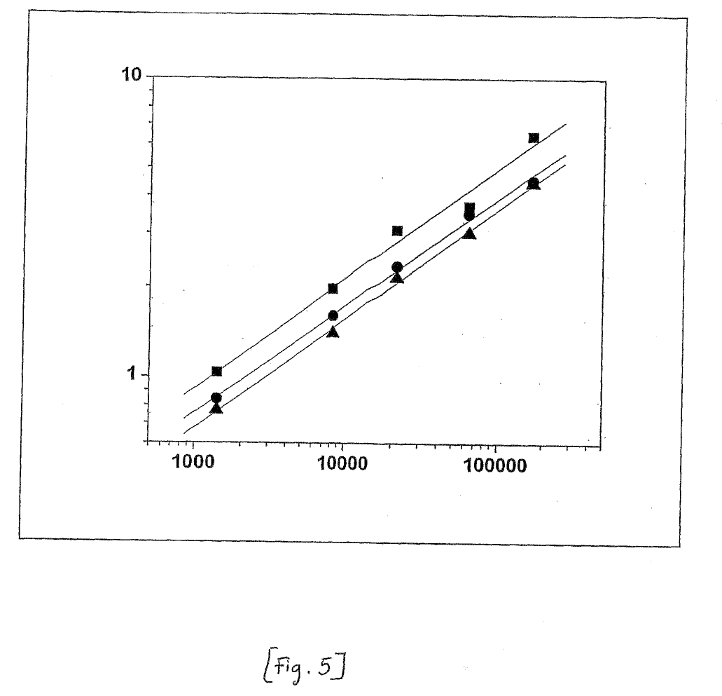

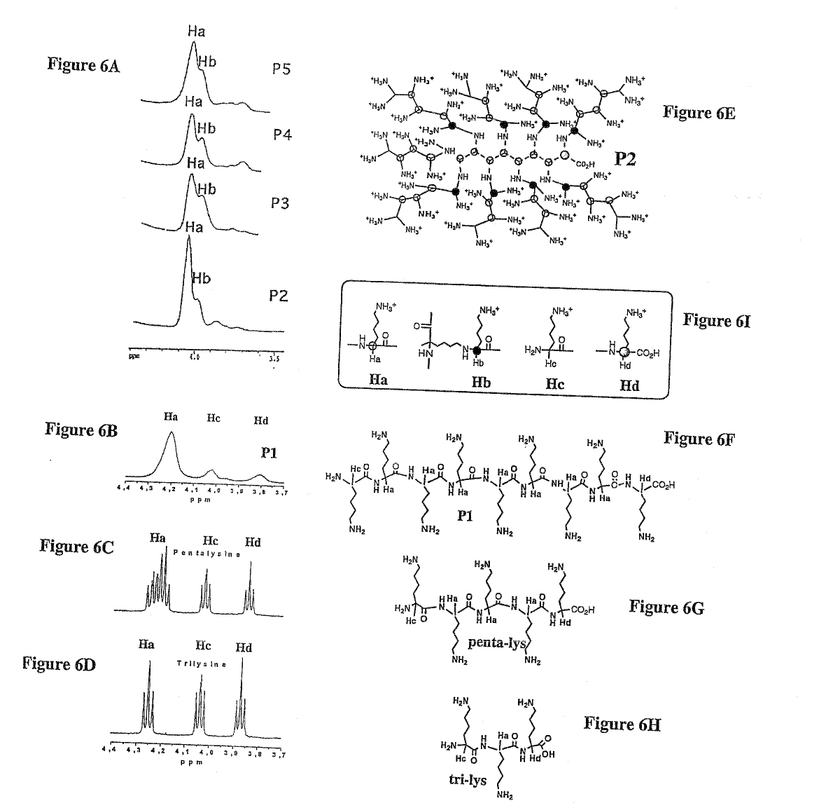

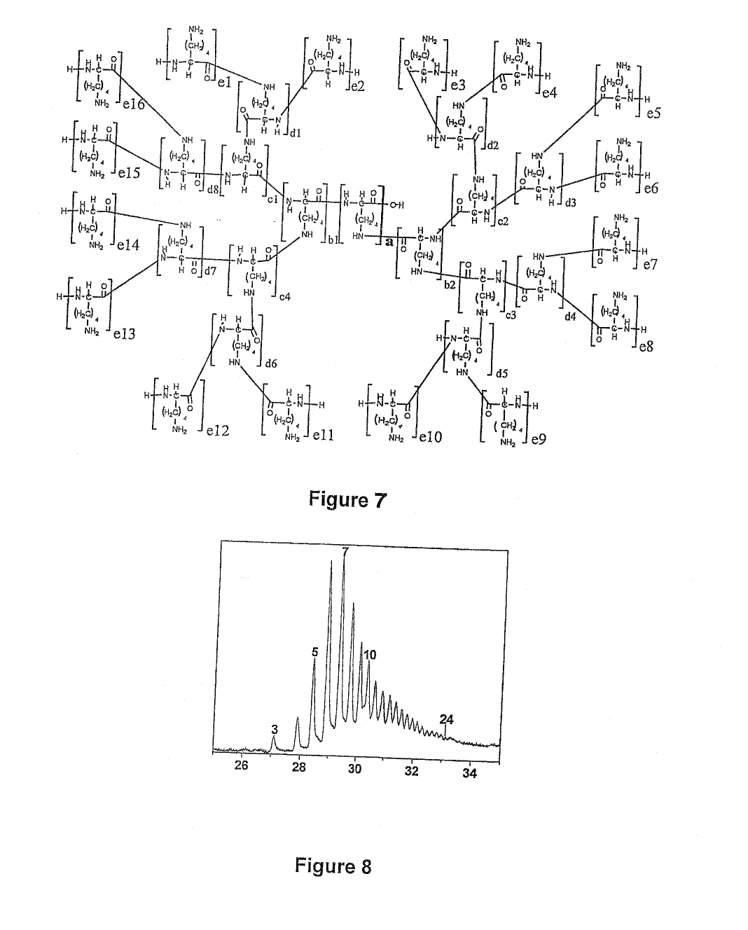

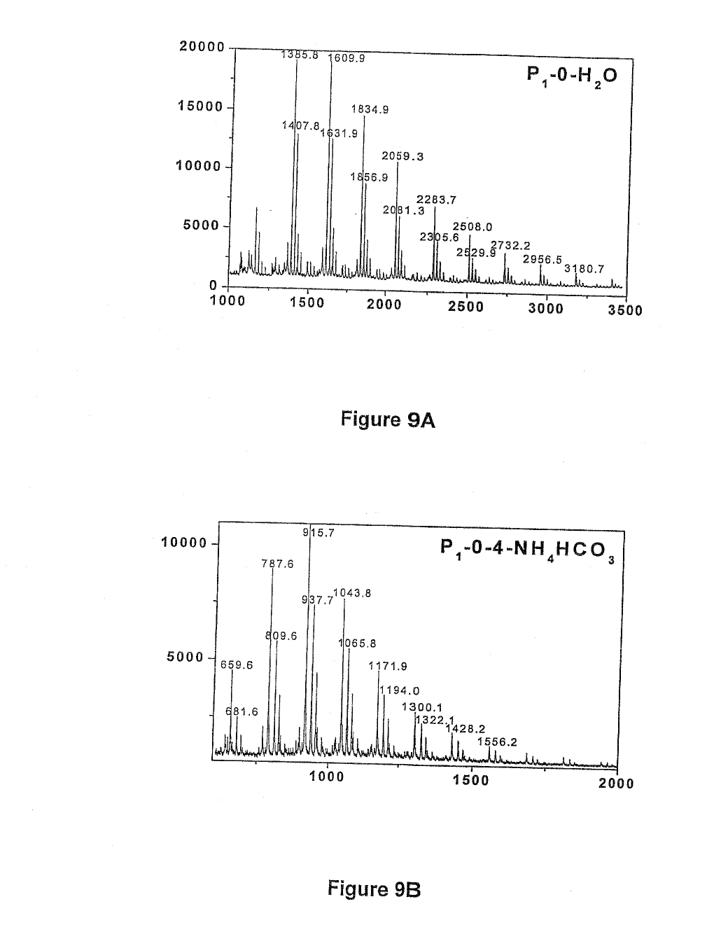

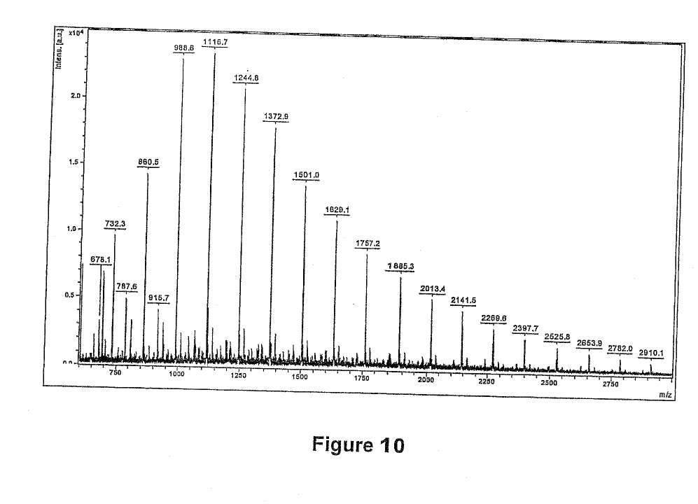

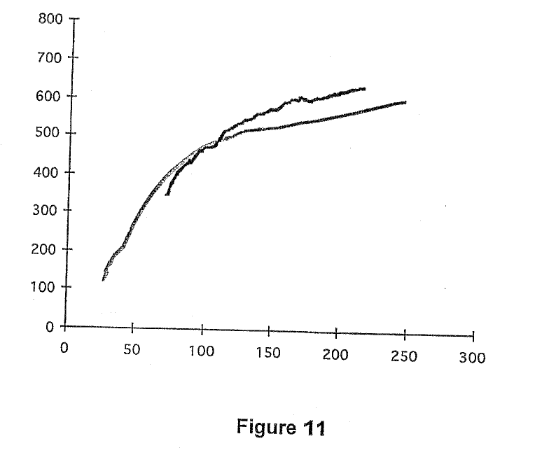

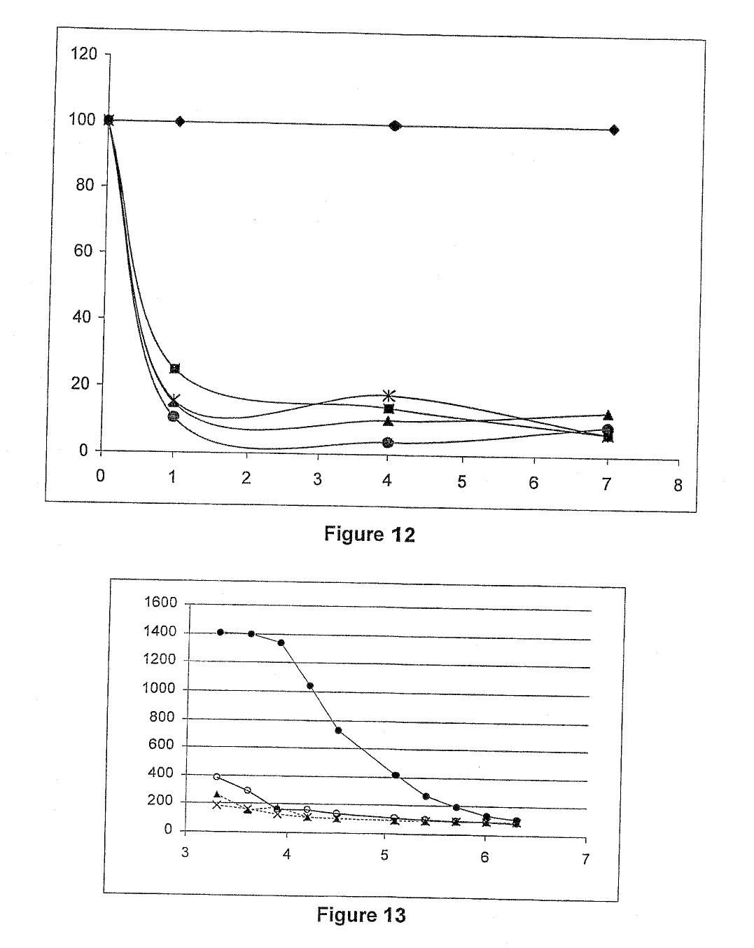

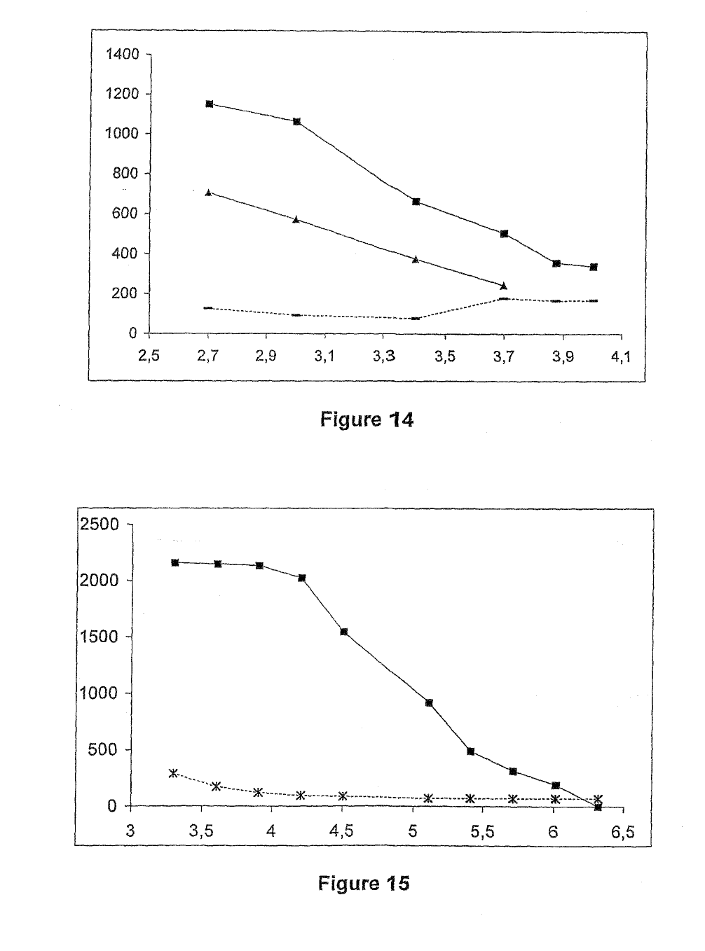

The variation in the molar mass of the GLDs is represented in FIGS. 2A-2B on a linear scale (FIG. 2A) or on a semi-logarithmic scale (FIG. 2B). By way of comparison, the expected (theoretical) molar weights are also shown for polylysine dendrimers primed either with a monoamine (dendrimer 1, with a benzhydrylamine core, [5, 33]), or with a diamine primer (dendrimer 2 primed with a 1,4-diaminobutane core, [7]. It is interesting to note that, in all cases (including the GLDs), the growth of the molar mass is exponential. Thus the molar mass between two successive generations is multiplied by the same factor. However in the case of the GLDs, the multiplication factor is 3.2 (e^{1.162}), against 2.12 for the dendrimer 2, and only 1.95 for the dendrimer 1. The molar weights of the GLDs have, as a result, a law of exponential growth, characteristic of the dendrimers, however, the increase in the molar mass takes place for the GLDs in a much more rapid manner than for the dendrimers of the same type.

It will be noted that the polydispersity indices are relatively low, comprised between 1.2 (generation 1) and 1.46 (generation 5), which demonstrates that the proposed method of synthesis makes it easily possible to obtain compounds of controlled polydispersity.

The results of the calculation of the average number of monomers incorporated by potential primers (α or ε amine function) during the passage from a generation i to a generation i+1 are given in Table 4. This result is consistent with the results for intrinsic viscosity which provide a minimal density (maximal intrinsic viscosity) for a dendrimer of functionality 3 in generation 4 (see Example 9).

### Example 8

### Determination of the Hydrodynamic Ranges (or Diffusion Coefficients) of the GLDs

In order to find the characteristic dimension of the GLDs in solution, the hydrodynamic ranges were determined from the molecular diffusion coefficients by using the Stoke-Einstein ratio:

$${D}_{m} = \frac{kT}{6 π η {R}_{h}} \qquad ( 1 )$$

where η is the viscosity of the medium, k the Boltzmann constant and T the temperature in K. In order to determine Dₘ, two experimental methods were used: quasi-elastic or static light diffusion (SLD) (coupled or not coupled with SEC), and the Taylor diffusion method (TDA). This latter method makes it possible to measure the diffusion coefficient of a species from the widening of a solute band under the influence of a hydrodynamic flow the speed profile of which is known to be dispersive (parabolic Poiseuille profile).

The TDA measurements were carried out using a capillary electrophoresis appliance, using silica capillaries of 50 μm i.d. (60 cm×50 cm up to the detector) the internal surface of which was previously modified by grafting a polycation (poly(diallyl-dimethyl-ammonium)) in order to limit the adsorption of the GLD solutes on the surface of the capillary. Dispersion of the solute band under the influence of the Poiseuille flow was measured using a UV spectrophotometer. The diffusion coefficient was then determined according to the method originally described by Taylor, then repeated by Bello et al. [34]. In parallel to the TDA method, measurements were carried out by SLD, using a zetasizer (Malvern) device. Finally, it was also possible to obtain Rₕ measurements from the 3D-SEC (Viscotek). All of the numerical data for Rₕ is shown together in Table 3.

FIG. 3 shows the increase in the GLD hydrodynamic ranges as a function of generation, in buffer 2 and for the three experimental methods used. It appears that the values obtained by TDA are less than those obtained by SLD or by 3D-SEC. This difference is probably due to the presence of aggregates in the sample which even in a small proportion, tend to increase the Rₕ values obtained by SLD. In fact, the aggregates of high molecular weight represent an important contribution to the diffused light signal (which varies with the molar mass to the power 6), which leads to an overvaluation of the Rₕ value corresponding to the unimers [35]. In 3D-SEC, the partial separation of the aggregates in relation to the unimers limits this effect, which can explain why the 3D-SEC values are closer to the TDA values. In conclusion, the values obtained by TDA appear to be the closest to the real values, as the contribution of the aggregates in the case of a detection by UV absorption is proportional to their mass concentration which is relatively low.

The influence of the pH of the buffer (buffer 1 vs buffer 3), at an ionic force which is more or less constant (of the order of 0.5 M), shows that at pH 7.0 the GLDs have a hydrodynamic range which is significantly lower than at pH 4.5 (FIG. 4). This result is logical in view of the deprotonation of the amine functions which must start to be effective at pH 7.0, thus limiting electrostatic repulsions. This pH effect should be even greater at a lower ionic force and/or for pH values above 7. The addition of 3% acetonitrile to buffer 1 also tends to reduce the hydrodynamic range, but to a moderate extent. This could be explained by a reduction in the quality of the solvent.

In conclusion, the GLDs have a hydrodynamic range which varies quasi-linearly with the number of generations. Once again, a behaviour similar to that of dendrimers is observed. Moreover, the increase in the pH and the addition of acetonitrile tend to reduce their hydrodynamic ranges. It will also be noted that the TDA method appears to be the most suitable method (and the most repeatable, with relative standard deviations of the order of 2-3% as against 10-20% for the SLD) for measurement of Rₕ.

### Example 9

### Determination of the Intrinsic Viscosity

The intrinsic viscosity [η] is a variable inversely proportional to the density of the molecule in the solvent. This variable consequently depends on the molecular weight and the volume (or hydrodynamic range) of the molecule. It is determined by viscosity measurements, by extrapolating the reduced or inherent viscosity at nil concentration. The 3D-SEC made it possible to obtain values for intrinsic viscosity. These are given in Table 3. It was interesting to note that [η] passes through a maximum for generation 4 as predicted by the theory for a dendrimer of functionality 3.

A study starting from the hydrodynamic ranges and the Van der Waals (V_{w}) volumes makes it possible to show that the density of the GLDs develops in the same way as for a dendrimer of functionality 3. The Van der Waals volume represents the volume actually occupied by the atoms (without taking into account the solvatation phenomena). It can be calculated [36] by a sum of increments, each increment corresponding to the volume of a atom (tabulated as a function of the nature of the neighbouring atom or atoms). For the polylysine of degree of polymerization n, the Van der Waals volume is given by the following relation:

V_{w}(nm³)=0.1226×DPₙ+18.6 10⁻³ (2)

Then it is possible to calculate the free-volume fraction f according to the equation:

f=1−(V_{w}/Vₕ) (3)

Table 5 shows the values for V_{w}, Vₕ and f for P1 and the 4 GLD generations. A maximum of f for P4 is noted from these values.

Another proof of the dendritic behaviour of the GLDs is provided by FIG. 5 which represents the variation in the hydrodynamic range of the GLDs as a function of their molar mass on a bi-logarithmic scale. The exponents obtained during the linearization of these data are of the order of 2.8, which is consistent with the values obtained for trifunctional dendrimers. These exponents are good indicators of the branching rate of the dendritic structures, knowing that a value of 3 corresponds to a maximum branching structure.

### Example 10

### Analysis of the Grafting Rate of the GLDs

The term grafting rate of a polylysine dendrimer of generation n (Pn), denotes the percentage of the ε-NH₂ functions which have reacted in relation to the total degree of polymerization of this product of generation n. (DPn). The grafting rate of the GPLDs of the invention are shown in the following Table 6.

TABLE 6
Principal features of the GLDs of the invention,
in particular branching points and grafting rate.

| | Mn (g/mole) by EC for | | | |
|---|---|---|---|---|
| | P1, by SEC-double | | Branching points | |
| Poly-L-lysines | detection for P2 to P5, | | determined by the | Grafting rate |
| of the invention | and by extrapolation for P6 | DPn | ¹H NMR ratio (Hb/Ha) | in % |
| P1 | 1,450 | 8 | (Core of the materials of this invention) | |
| | | | Linear polylysine of 8 units (Mn = 8) | |
| | | | 8 | 8/8 = 100% |
| | | | determined from the ratio | |
| | | | Hb/Ha = 0.25 of P2 | |
| P2 | 8,600 | 48 | 39 | 39/48 = 81% |
| | | | determined from the ratio | |
| | | | Hb/Ha = 0.60 of P3 | |
| P3 | 22,000 | 123 | 98 | 98/123 = 80% |
| | | | determined from the ratio | |
| | | | Hb/Ha = 0.46 of P4 | |
| P4 | 65,300 | 365 | 237 | 237/365 = 65% |
| | | | determined from the ratio | |
| | | | Hb/Ha = 0.40 of P5 | |
| P5 | 172,000 | 963 | 614 | 614/963 = 64% |
| | | | determined from the ratio | |
| | | | Hb/Ha = 0.42 of P6 | |
| P6 | 439,800 | 2457 | / | / |

The grafting rates given in Table 6 were obtained by a progressive analysis of the construction of the grafted dendrimers starting from generation 1 to generation n. The grafting rate of P1 to P2 is calculated from the experimental values of the degrees of polymerization; DP=8 for P1, DP=48 for P2 and the experimental ratio Hb/Ha=0.25 (FIGS. 6A-6I) On the basis of these values, the variation of DP is 40. Considering that the maximum number of reactive functions is 8ε NH₂ and 1 α NH₂, that in carbonated water and at pH 6.5 the reactivity of the α NH₂ functions (pK=7.2) was only slightly greater than that of the ε NH₂ functions (pK=11.2), it is deduced that the 9 NH₂s of the P1 incorporate 4.4 monomers on average. Thus, in P2, the maximum Hb number is 8, the Ha number 30 (40−9 He+1 Hd which in NMR have different resonance fields). These values lead to an Hb/Ha ratio=8/30=0.26 which is identical to the experimental ratio. This indicates that the 8ε NH₂s of P1 as well as the α-NH₂ function of P1 reacted in order to produce P2, which shows that 8 ENH₂s out of a total of 8 εNH₂s of P1 reacted to produce P2. Thus the grafting rate of the P1 was 8/8=100%.

After release, P2 carries 40 εNH₂s and 9 αNH₂s. The grafting rate of P2 to pass to P3 is calculated from the experimental values of DP=48 for P2, DP=123 for P3, the variation of DP=75 and from the experimental ratio Hb/Ha=0.60 (FIGS. 6A-6I).

The only way to satisfy this experimental value is to accept that the number of reactive εNH₂s of P2 is 31 out of a possible 40 (9 εNH₂s are therefore non-reactive). The 75 Lys (variation of DP) can then be distributed as follows: 10 εNH₂ each extend from one lysine, 21 εNH₂s from two lysines, 4 αNH₂s from two lysines and 5 αNH₂s from three lysines, the latter to take into account the reactivity of the αNH₂s which is slightly greater than that of the εNH₂s. This solution corresponds to an Hb number=31+8=39, with 64 Ha and 20 Hc+1Hd, which gives a ratio of Hb/Ha=39/64=0.60 equal to the experimental ratio. This shows that 39 εNH₂s out of a total of 48 εNH₂s of the P2 have reacted in order to produce the P3, and that the grafting rate of the P2 is 39/48=81%.

A similar reasoning based on the experimental ratios Hb/Ha makes it possible to obtain the grafting rate of the different P3, P4, P5 shown in Table 6.

These grafting rates show that the grafted lysine dendrimers of the present invention are very different, and in particular much denser than the branched polylysines described by Klok et al. [3] (See Table 1). The high grafting density of the compounds of the invention, produced directly by the reactivity of the NCAs on the α and ε NH₂ functions in bicarbonated water at a pH of approximately 6.5 is an additional feature of the present invention.

TABLE 2
Definition of the buffers and eluents used for determination
of the physico-chemical variables of the GLDs

| | |
|---|---|
| Buffer 1 | H₂PO₄⁻Na⁺50 g/L, pH 4.5 in water |
| | (ionic force 0.61 M) |
| Buffer 2 | Buffer 1 + 3% acetonitrile (by weight) |
| Buffer 3 | Physiological buffer: 38 mM H₂PO₄⁻Na⁺+ |
| | 38 mM HPO₄²⁻, 2Na⁺+ 9 g/L NaCl, adjusted |
| | to pH 7.0 (ionic force 0.48 M) |

TABLE 3
Table showing all the physico-chemical values determined on the GLDs

| | | P1 | | P2 | | P3 | | P4 | | P5 | | Method: |
|---|---|---|---|---|---|---|---|---|---|---|---|---|
| Buffer 1 | Mₙ (g/mol) | 1 450 | | 8 600 | | 22,000 | | 65 300 | | 172 300 | | 2D-SEC |
| | DPn | 8 | | 48 | | 123 | | 365 | | 963 | | |
| | I | 1.2 | | 1.40 | | 1.46 | | 1.36 | | 1.46 | | |
| | dn/dC | 0.112 | | 0.127 | | 0.134 | | 0.137 | | 0.136 | | |
| | (mL/g) | | | | | | | | | | | |
| | Rₕ (nm) | 1.1 | (3%) | 2.1 | (0.9%) | 3.2 | (2%) | 3.92 | (5%) | 6.74 | (2%) | TDA |
| | Rₕ (nm) | 1.18 | (17%) | 2.33 | (9%) | 3.41 | (8%) | 6.31 | (8%) | 8.38 | (8%) | SLD |
| Buffer 2 | Rₕ (nm) | 0.95 | (2.4%) | 1.82 | (1.1%) | 2.64 | (0.2%) | 3.94 | (2.8%) | 5.13 | (0.8%) | TDA |
| | Rₕ (nm) | 1.1 | (26%) | 2.21 | (8%) | 3.73 | (12%) | 5.98 | (6%) | 8.5 | (13%) | SLD |
| | Mₙ (g/mol) | | | 8 600 | | 22,000 | | 65 300 | | 172 300 | | 3D-SEC |
| | Rₕ (nm) | | | 1.79 | | 2.95 | | 4.95 | | 7.99 | | |
| | [η] (dL/g) | | | 5.6 | | 6.6 | | 7.1 | | 7.1 | | |
| Buffer 3 | Rₕ (nm) | 0.74 | (11.4%) | 1.34 | (4.6%) | 2.05 | (0.63%) | 2.87 | (2.2%) | 4.24 | (0.26%) | TDA |
| | Rₕ (nm) | 1.01 | (ND) | 1.69 | (ND) | 3.28 | (13%) | 5.89 | (15%) | 6.85 | (17%) | SLD |

I represents the polymolecularity index. DPₙ represents the average degree of polymerization. The relative standard deviations are given in brackets. The number n of independent experimental determinations is 5. 2D-SEC: double detection steric exclusion chromatography (light diffusion and refraction index) under the experimental conditions described in FIG. 1; 3D-SEC: triple detection steric exclusion chromatography (light diffusion, viscosimetric detector and refraction index). Experimental conditions of the 3D-SEC: 5% NaH₂PO₄ buffer+3% Acelonitrile by volume, pH˜4. Flow rate: 0.6 mL/min. Volume injected. 100 μL. Superose 12 HR 10/30 column. Temperature. 28° C.

TABLE 4
Calculation of the average number of monomers incorporated by
potential primers (α or ε amine function) during the
passage from a generation i to a generation i + 1

| | | | | average |
|---|---|---|---|---|
| Passage | | | | number |
| from | Mₜ₊₁- | | number of | of monomers |
| generation | Mₜ | Nₜ₊₁- | primers | incorporated/ |
| i to i + 1 | (g/mol) | Nₜ | at i | primer |
| 1/2 | 7 150 | 40 | 9 | 4.4 |
| 2/3 | 13 400 | 75 | 48 | 1.55 |
| 3/4 | 43 300 | 242 | 123 | 1.96 |
| 4/5 | 107,000 | 598 | 366 | 1.64 |

TABLE 5
Hydrodynamic volumes, Van der Waals volumes
and free-volume fraction of the GLDs in relation
to the generation number in buffer 2

| GLD | Vh (nm³) | VW (nm³) | f |
|---|---|---|---|
| 1 | 3.59 | 1.0 | 0.721 |
| 2 | 25.3 | 5.9 | 0.766 |
| 3 | 77.1 | 15.1 | 0.804 |
| 4 | 256 | 44.8 | 0.825 |
| 5 | 565 | 118 | 0.791 |

The degrees of polymerization taken into account for the VW calculations are those given in Table 2.

### Example 11

### Synthesis of Triblock Copolymers

### Grafted poly(L-lysine)-block-poly(ethylene glycol)-block-grafted poly(L-lysine)

1/ 0.5 g of N^{ε}-TFA-L-Lysine-NCA is synthesized.

2/ 5 ml of an aqueous solution 0.1 M of HCO₃Na maintained at 0° C., in which 0.42 g of poly(ethylene glycol)-α,ω-diamine of 3400 Da (Shearwater Corporation) has been dissolved, is added to the crude product of the reaction.

3/ After 30 min, the white precipitate of grafted {linear poly(TFA-L-lysine)-block-poly(ethylene glycol)-block-linear poly(TFA-L-lysine)}, formed in the medium, is recovered by filtration or centrifugation.

4/ This precipitate, placed in 10 ml of aqueous ammonia solution at pH 12 for 15 hours at 40° C., loses the protective TFA groups of the side chains of the units added during the polymerization and becomes soluble in water.

5/ The volume of the solvent is reduced by half in a rotary evaporator.

6/ After lyophilization, 0.45 g of a {linear poly(L-lysine)-block-poly(ethylene glycol)-block-linear poly(L-lysine)} of generation 1 is obtained. Its polydispersity is 1.07 (measured by SEC). Its average mass measured by LS is 15,500 Da.

By repeating this reaction, and by using as primer 0.15 g of the product obtained during the previous reaction, 0.3 g of a {grafted poly(L-lysine)-block-poly(ethylene glycol)-block-grafted poly(L-lysine)} of polydispersity 1.3 and of average mass equal to 23,800 Da measured by SEC-double detection (light diffusion refraction index) was obtained.

### Example 11A

### Synthesis of Fluorescent GLD of Generation 1 (P1)

5 ml of a 0.1 M aqueous solution of HCO₃Na maintained at 0° C. in which 5 mg of aminofluorescein is dissolved, is added to 500 mg of N^{ε}-TFA-L-Lysine-NCA of Example 1. The amine function of the aminofluorescein reacts on the carbon 5 of the NCA and forms H-(lysine N^{ε}-TFA)-NH-fluorescein, which in turn, reacts on other NCAs to form H-(lysine-N^{ε}-TFA)ₙ-NH-fluorescein which precipitates with a DPₙ equal to 8.

The precipitate treated like that of Example 1 leads to 240 mg of linear poly-L-Lysine of formula (⁺H₂(Lys)ₙ-NHC₂₀H₁₁O₅) with n comprised between 2 and 20, characterized by MALDI-TOF as shown in FIG. 10. Excited at 490 nm, this material fluoresces in a 0.1 M solution of sodium bicarbonate at pH 8.5 with a maximum wavelength at 517 nm and a detectability threshold of less than 10⁻³ mg·L⁻¹ by an SLM-Aminco MC 200 monochromator spectrofluorimeter in a 1 cm cell. This maximum emission wavelength is to be compared with that of the free aminofluorescein (513 nm) under the same conditions. To observe the fluorescence of the dendrimer, 5 mg of this material is dissolved in 10 ml of a 0.1 N solution of bicarbonate. 100 μl of this last solution are diluted to 1% by the 0.1 N solution of bicarbonate. This solution was excited at 490 nm. The fluorescence emission was observed at a maximum wavelength of 517 nm. By successive dilutions the detectability threshold of an SLM-Aminco MC 200 monochromator spectrofluorimeter using a 1 cm cell is reached when the concentration is equal to 10⁻³ mg·L⁻¹. The maximum emission wavelength of 517 nm is to be compared to that of the free aminofluorescein (513 nm) under the same conditions.

### Example 11B

### Synthesis of Fluorescent GLD of Generation 2 (P2)

This synthesis was carried out in a similar way to that described in Example 2 by using as primer the product of the reaction described in Example 11A. The product obtained fluoresces at 517 nm (excitation 490 nm). Its detectability threshold is of the order of 10⁻³ mg·L⁻¹ under conditions identical to those of Example 11A.

### Example 11C

### Synthesis of Fluorescent GLD of Generation 3 (P3)

This synthesis is carried out in a similar way to that described in Example 3 by using as primer the product of the reaction described in Example 11B. The product obtained fluoresces at 519 nm (excitation 490 nm). Its detectability threshold is of the order of 10⁻² mg·L-1 under conditions identical to those of Example 11A.

### Example 11D

### Synthesis of (Red) Fluorescent GLD of Generation 1 (P1)

200 mg of a generation 11 linear Poly-N-TFA-L-Lysine (P1) of DPₙ 8, obtained as in Example 1, is dissolved in 2 ml of anhydrous dimethylformamide (DMF). 5 mg of rhodamine sulphochloride is added to this solution. The homogeneous medium obtained is maintained at 25° C. for 24 hours under magnetic stirring. 10 ml of an aqueous solution of sodium bicarbonate 0.1N was then added to the reaction medium. A light red precipitate immediately forms in the medium. This precipitate is separated by centrifugation at 5000 rpm for 10 min. It is then again placed in suspension in 10 ml of a 0.1 N solution of sodium bicarbonate and centrifuged 10 times to remove the unreacted rhodamine. The last washing waters are colourless, while the last precipitates remain a light red colour, showing that the rhodamine is well fixed onto the protected P1. The precipitate P1 is then placed in 50 ml of a 1 N aqueous solution of ammonia/methanol 50/50 for 15 hours to release the epsilon functions from their TFA protective groups. The reaction medium is then reduced by half under vacuum to remove the methanol and the ammonia. After lyophilization, 150 mg of a light red product is obtained. Its bright red fluorescence characterizes this new first generation dendrimer marked with rhodhamine (Texas red).

### Example 12

### Modifications to the Free Amine Functions of the GLDs

1 gram of 3,5-dimethylester-dicarboxyphenyl isothiocyanate is added to a gram of GLD P2 of Mn 8 600 Da in solution in 50 cm³ of water-acetonitrile 80/20 solvent. The resulting solution is maintained at 40° C. for 10 hours. Two grams of ammonium hydrogen carbonate is added to this solution. After 5 hours of contact at 40° C., the medium is dialyzed and lyophilized. 1.50 g of GLD P2 is obtained, in which each amine function is bound by a thiourea function to a sodium phenyl-3,6-dicarboxylate. The NMR in D₂O of the product obtained shows the presence of the GLD signals associated with those of the aromatic group.

### Example 13

### Ionic Fixing of the GLDs on Support

3 g of GLD P3 of Mn 22,000 Da are incubated with 24 g of Norit carbon (GAC 1240 PLUS) in 70 ml of sterile distilled water for 24 hours. The carbon is then washed with 250 ml of sterile distilled water. After evaporation of the latter, 1.82 g of P3 is recovered, which shows that 1.18 g of P3 is absorbed on 24 g of Norit carbon. The activity of 1 g of this carbon on which 49 mg of P3 is absorbed is tested on Micrococcus lysodeikticus, comparing it to that of a control support without P3.

### Example 14

### Covalent Fixing of the GLDs on Support

A hexamethylene-diisocyanate [OCN—(CH₂)₆—(NHCONH—(CH₂)₆)ₙ—NCO] oligomer is used to fix the GLDs covalently onto the support resins. Known quantities of GLD (P1, P2, P3) are mixed vigorously in approximately 5 g of oligomer so as to have variable percentages of GLD from 0 to 20%, and the mixture is plated on glass plates. After 48 hours, the resin obtained is ground until particles are obtained comprised between 0.1 and 1 mm, and it is washed copiously with 250 ml of acetone, 250 ml of water, 250 ml of methanol, 250 ml of ethyl ether and dried.

### Example 14A

### Covalent Fixing of P2 and of P3 on Activated Silica

25 mg of fluorescents GLDs obtained in the examples 11B and 11C are dissolved in 25 mL of a 0.1 N aqueous solution of HCO₃Na at 25° C. 1 g of silica grains of 50 microns, specific surface area (500 m²/g), carrying 1.1 meq of Si—(CH₂)₃NCO, is added to this solution, which is stirred by bubbling nitrogen through it. After 2 hours, the grains of silica are separated, washed in water and dried. 20 mg of these GLDs, measured by difference from the original solution, are thus fixed onto 1 g of support. The surface of these supports, observed with a fluorescence microscope, have the green fluorescence characteristic of fluorescein.

### Example 15

### Toxicity Study of the GLDs In Vivo

Experiments were carried out on rats of the Sprague-Dawley strain aged from 4 to 8 weeks (Bred by CER Janvier, Le Genest St-Isle).

A. Acute Toxicity:

5 rats receive an injection of 2 mL of a solution of GLD P3 (60 mg/ml) three times a day for 4 days. The results obtained are shown in the following Table 7:

TABLE 7
Acute toxicity study for a solution of GLD P3

| | | | |
|---|---|---|---|
| Weight day 1 | Weight day 4 | Var % | Var % |

| average | S.D. | average | S.D. | average | S.D. |
|---|---|---|---|---|---|
| 252 | 10 | 280 | 11 | 11.0 | 10.0 |

No toxic effect is visible.

B. Chronic Toxicity:

The rats receive a sub-cutaneous injection of 0.5 mL of GLD P3 (60 mg/ml) each day for 180 days. The control rats receive 0.5 ml of physiological saline (solvent of the solution). The weight development of the treated rats and the control rats is compared (FIG. 7). No change in the weight development nor any unwanted effect was observed.

These experiments emphasise that the grafted lysine dendrimer (GLD) P3 has no apparent toxicity characteristics: all the animals are alive and have a homogeneous and regular weight development, which can be superposed on that of the controls.

### Example 16

### Antitumor Activity of the GLDs

Cytotoxicity tests were carried out on myelomatous cells of mice with variable concentrations of GLDs P2, P3, P4 and P5. The results obtained are shown in the following Table 8.

TABLE 8
Comparative study of the cytotoxic activity of the polylysine
dendrimers of the invention on myeloma cells

| | Concentration μM | | | | | | | | | |
|---|---|---|---|---|---|---|---|---|---|---|
| | 50 | 25 | 12.5 | 6.25 | 3.125 | 1.56 | 0.78 | 0.39 | 0.195 | 0.09 |
| P2 (8,600 Da) | ++++ | ++++ | +++ | ++ | + | − | − | − | − | − |
| in mg/L | | | | 52 | | | | | | |
| P3 (22,000 Da) | ++++ | ++++ | ++++ | +++ | +++ | ++ | + | +/− | − | − |
| in mg/L | | | | | | 34 | | | | |
| P4 (65,300 Da) | ++++ | ++++ | +++ | +++ | ++ | + | +/− | − | − | − |
| in mg/L | | | | | 202 | | | | | |
| P5 (172,300 Da) | ++++ | ++++ | ++++ | +++ | ++ | ++ | + | +/− | − | − |
| in mg/L | | | | | | 263 | | | | |

++++ = cell clusters and cell lyses
++++ = agglutinated cell clusters and cell lyses
− = living cells identical to the control

These tests on myelomatous cells show that the GLDs are active on murine myeloma for concentrations equal to or greater than 34 mg/L depending on the GLD generation studied. The GLDs of high molar mass are the least active.

### Example 17

### Antifungal Activity of the GLDs

The antifungal tests were carried out on a filamentous fungus, Fusarium oxysporum (INRA Collection Saint Christol-lés-Alès, France) according to the technique of growth inhibition in a liquid medium described by Fehlbaum et al. [37].

80 μl of the spores of F. oxysporum (final concentration of 10⁴ spores·mL⁻¹) in suspension in the medium PDB (Potato Dextrose Broth, Difco) containing 0.1 mg of tetracycline, are added to 20 μl of the different dilutions of the dendrimers in microplates. The dendrimers are replaced by 20 μl of sterile water for the controls. The growth inhibition was observed under the microscope after 24 hours of incubation at 30° C. under stirring and quantified after measurement of the optical density at 48 hours. The minimum growth-inhibitory concentration (MIC) is assessed by serial dilutions (in doublets) of the molecules and defined as the lowest dendrimer concentration inhibiting growth.

The results of the antifungal tests are given in the table below.

TABLE 9
Comparative study of the antifungal activity of
the grafted lysine dendrimers of the invention:

| | Grafted lysine dendrimers | | | |
|---|---|---|---|---|
| Fungus: | P2 | P3 | P4 | P5 |
| Fusarium oxysporum | | | | |
| (INRA Collection St Christol lés Alès) | | | | |
| in μM | 22 | 1.25 | 0.54 | 0.36 |
| in mg/L | 183 | 27 | 35 | 61 |

(MIC values in μM and mg/L)

It can be noted overall that the MIC is equal to 27 mg/L for Fusarium oxysporum.

### Example 18

### Antibacterial Activity of the GLDs

The method used for the antibacterial tests is a modification of the technique of Hancock et al. [38]. Measurement of the antibacterial activity is carried out on 3 strains of Gram-positive bacteria: (Micrococcus lysodeikticus ATCC 4698, Bacillus megaterium ATCC 17748, Staphylococcus aureus ATCC 25293), and 4 strains of Gram negative bacteria: (Escherichia coli 363 ATCC 11775, Vibrio penaeicida, IFREMER Collection, Vibrio anguillarum Listonella anguillarum ATCC 14181, Vibrio alginolyticus ATCC 17749) by measurements of growth inhibition in a liquid medium.

A. In the Homogeneous Phase:

Briefly, 10 μl of the synthesis molecules (P2, P3, P4 and P5) are incubated in microplates with 100 μl of each bacterial suspension at the starting concentration D600=0.001, in PB medium (Poor Broth: bactotryptone 1%, NaCl 0.5% w/v, pH 7.5). The bacterial growth is measured at 600 mm after 24 hours' incubation at 30° C. under stirring. The minimum growth-inhibitory concentration (MIC) is assessed by serial dilutions (in doublets) of the molecules and defined as the lowest dendrimer concentration inhibiting growth of the bacteria.

The results of the antibacterial tests given in the table below demonstrate that the activity spectrum of the dendrimers P2 to P5 is broad, in fact these molecules are active on both Gram + and Gram − bacteria.

TABLE 10
Comparative study of the antibacterial activity of the GLDs

| | Grafted lysine dendrimers | | | |
|---|---|---|---|---|
| Microorganisms | P2 | P3 | P4 | P5 |
| Gram+ | | | | |
| M. lysodeikticus ATCC 4698 | | | | |
| in μM | 1.56 | 0.78 | 1.56 | 0.39 |
| in mg/L | 13 | 17 | 100 | 66 |
| B. megaterium ATCC 17749 | | | | |
| in μM | 0.39 | 1.56 | 1.56 | 0.78 |
| in mg/L | 3 | 34 | 100 | 132 |
| Staphylococcus aureus | | | | |
| ATCC 25293 | | | | |
| in μM | >12.5 | 3.125 | 0.78 | 0.39 |
| in mg/L | 104 | 67 | 50 | 66 |
| Gram− | | | | |
| E. coli 363 ATCC 11775 | | | | |
| in μM | 2.8 | 1.56 | 33.3 | 22.2 |
| in mg/L | 23 | 33.5 | 2131 | 3751 |
| Vibrio penaeicida | | | | |
| IFREMER Collection | | | | |
| in μM | >50 | 25 | 3.125 | 1.56 |
| in mg/L | 417 | 537 | 200 | 264 |
| Vibrio anguillarum | | | | |
| in μM | 22.2 | 2.8 | <1.9 | <1.9 |
| in mg/L | 185 | 60 | 121 | 321 |
| Vibrio alginolyticus | | | | |
| ATCC 17749 | | | | |
| in μM | 9.9 | 2.8 | <1.9 | <1.9 |
| in mg/L | 83 | 60 | 121 | 321 |

(MIC values in μM and mg/L)

The antimicrobial activity expressed in μM or mg/L shows a wide variability from one dendrimer to another and depending on the bacterial strain studied. It can be noted overall that the MICs vary, using in each case the most active generation of GLD, from 3 to 50 mg/L for the Gram +, and from 23 to 200 mg/L for the Gram −.

B. In the Heterogeneous Phase:

The antibacterial activity of the GLD dendrimers fixed ionically or covalently onto a support was tested.

1. Ionic Fixing:

The fixing was carried out as described in Example 13. Then, the supports (1 g) are washed 5 times with 20 ml of sterile water and dried. The products obtained are placed in contact with 2 ml of bacterial suspension (Micrococcus lysodeikticus) for 24 hours. After this incubation period, 100 μL of supernatant is removed and plated on an LB agar dish and incubated for 24 hours at 37° C. The number of colonies observed (or not observed) shows the activity of the supports used. The results are shown in the following table.

TABLE 11
Antibacterial activity of GLDs fixed ionically onto support:

| | Observation |
|---|---|
| Control | Dish infested |
| Carbon + P3 | Absence of colonies |

These results indicate that the GLDs also perform an antimicrobial activity when fixed ionically onto a support.

As the ionic fixings are not necessarily permanent, the antimicrobial activity of the covalently fixed GLDs was also tested.

2. Covalent Fixing:

The fixing was carried out as described in Example 14. Then, 0.5 g of each of the materials is placed in contact with 2 ml of bacterial suspension (Micrococcus lysodeikticus), containing 2.106 bacteria, for 24 hours. After this incubation period, 25 μL of supernatant is removed and placed on 75 μL of LB medium. This mixture is plated on an LB agar dish and incubated at 37° C. After 96 hours of incubation the reading gives the results shown in the table below.

TABLE 12
Antibacterial activity of GLDs fixed covalently onto support:

| Materials | Number of colonies to J + 3 |
|---|---|
| resin (control) | Dish infested |
| resin + 2% P1 | 300 |
| resin + 2% P2 | 0 |
| resin + 2% P3 | 35 |
| resin + 1% P3 | 65 |
| resin + 0.5% P3 | >300 |

The results show that the GLDs fixed covalently onto support retain an excellent antibacterial activity at concentrations greater than or equal to 1%. Complementary studies must be carried out with finer grinding and with new resins.

### Example 19

Activities of the GLDs on Pseudomonas aeruginosa

A. In the Homogeneous Phase:

Suspensions of Pseudomonas aeruginosa with 10,000 bacteria/ml were placed in contact with solutions of P2 and P3 at 1, 10 and 100 mg/L. At 6, 24 and 96 hours controls were carried out by culturing on cetrimide.

The results shown in the following table show the total inhibition of Pseudomonas by solutions of P3 at 100 mg/L.

TABLE 13
Effect of the GLDs in homogeneous phase on a suspension of 10,000 P. aeruginosa/ml

| | control | | | | | | |
|---|---|---|---|---|---|---|---|
| | PBS + | P2 | P2 | P2 | P3 | P3 | P3 |
| Time | suspension | at 1 mg/L | at 10 mg/L | at 100 mg/L | at 1 mg/L | at 10 mg/L | at 100 mg/L |
| T = 0 | >300 | / | / | / | / | / | / |
| T = 6 h | >300 | 0 | 0 | 0 | 0 | 0 | 0 |
| T = 24 h | Dish | Dish | 1 | 1 | 108 | 0 | 0 |
| | infested | infested | | | | | |
| T = 96 h | Dish | Dish | 300 | 150 | Dish | 250 | 0 |
| | infested | infested | | | infested | | |

(filtration of 100 μl on membrane + culturing on cetrimide)

B. In Supported Phase:

The Pseudomonas aeruginosa were exposed to hexamethylene-diisocyanate resins (as synthesized previously) containing 10% by weight of P3 (P3S) for variable periods of time. At 6, 24 and 96 hours controls were carried out by culturing on cetrimide. The results shown in the following table show the total inhibition of Pseudomonas by supports of this type on suspensions of 1,000 Pseudomonas/ml.

TABLE 14
Effect of the GLDs in supported phase on suspensions of P. aeruginosa:

| | Suspension of 10,000 Pseudomonas/ml | | Suspension of 1,000 Pseudomonas/ml | |
|---|---|---|---|---|
| | (filtration of 100 μl on membrane + | | (filtration of 100 μl on membrane + | |
| | culturing on cetrimide) | | culturing on cetrimide) | |
| | Control PBS + | | Control PBS + | |
| Time | suspension | P3S at 10 g/L | suspension | P3S at 10 g/L |
| T = 0 | 53 | / | 3 | / |
| T = 6 h | 83 | 1 | 9 | 0 |
| T = 24 h | Dish infested | 0 | 8 | 0 |
| T = 96 h | Dish infested | Dish infested | Dish infested | 0 |

### Example 20

Activity of the GLDs on Legionella pneumophila

A. In the Homogeneous Phase:

Suspensions of Legionella pneumophila 10,000/ml were placed in contact with solutions of P2 and P3 at 10 and 100 mg/L. At 24, 96 and 168 hours, controls were carried out by culturing on GVPC.

The results shown in the following table and in FIG. 12 show that Legionella pneumophila is more sensitive to P2 than to P3.

TABLE 15
Effect of the GLDs in homogeneous phase on
a suspension of Legionella pneumophila

| | | | | | |
|---|---|---|---|---|---|
| | Control PBS + | | | | |
| Time | susp | P2 at 10 mg/L | P2 at 100 mg/L | P3 at 10 mg/L | P3 at 100 mg/L |

Suspension at 10,000 legionella/ml (plating of 100 μl on GVPC)

| | | | | | |
|---|---|---|---|---|---|
| D0 | 210 | 210 | 210 | 210 | 210 |
| D + 1 | 180 | 19 | 27 | 45 | 28 |
| D + 4 | 78 | 3 | 8 | 11 | 14 |
| D + 7 | 45 | 4 | 6 | 3 | 3 |

Relative inhibiting effect observed with respect to the control

| | | | | | |
|---|---|---|---|---|---|
| D0 | 100 | 100 | 100 | 100 | 100 |
| D + 1 | 100 | 10.6 | 15.0 | 25.0 | 15.6 |
| D + 4 | 100 | 3.8 | 10.3 | 14.1 | 17.9 |
| D + 7 | 100 | 8.9 | 13.3 | 6.7 | 6.7 |

A PCR control was carried out.

Briefly, after 96 hours of contact with dendrimers P2 and P3 at 10 and 100 mg/L, 5 ml of bacterial suspension is filtered on a 0.45 μm polycarbonate membrane to extract the cells present (the cell residues are thus removed). The cells are recovered on the filter, their DNA is extracted by thermal and chemical shocks. 1 ml is purified by ultrafiltration and quantified by PCR amplification. The results are shown in the following table.

TABLE 16
quantification of the DNA of the suspensions
of L. pneumophila in the presence of P2 or P3

| | Suspension extracted, purified + PCR | |
|---|---|---|
| | Results for 10 ml of suspension | Relative results |
| Control 10000/ml | 2.94 · 10⁵ | 100 |
| P2 (10 mg/ml) | 2.08 · 0⁴ | 7 |
| P2 (100 mg/ml) | 2.68 · 10⁴ | 9 |
| P3 (10 mg/ml) | 7.94 · 10⁴ | 27 |
| P3 (100 mg/ml) | 6.46 · 10⁴ | 22 |

Thus, the PCR analysis shows:
that the presence of P2 or P3 allows the removal from the culture medium in 96 hours of respectively more than 90%, or between 70 and 80% of bacteria relative to the control. The activity of P2 was greater than that of P3 on Legionella pneumophila.
that P2 or P3 at lethal concentrations causes a bacterial degradation by perforation of the cell walls and release of their DNA.

B. In Supported Phase:

Hexamethylene-diisocyanate resins containing 2% by weight of P1 (P1S) and 20% by weight of P3 (P3S) are obtained as previously.

Test No. 1:

Tube 1: The control is constituted by 9 ml of PBS+1 ml PBS containing 10,000 Legionella pneumophila.

Tube 2: 9 ml of PBS+1 ml PBS containing 10,000 Legionella pneumophila+0.1 g of P3S.

The results are shown in the following table:

TABLE 17
quantification of the DNA of suspensions
of L. pneumophila in the presence of P3S

| | | Suspension extracted, purified + PCR | Relative |
|---|---|---|---|
| | | results in 10 ml of susp. | result |
| Tube 1 | Control | 7.36 · 10⁴ | 100 |
| | 10000/ml | | |
| Tube 2 | P3S | 7.36 · 10³ | 10 |

Test No. 2:

Tube 1: The control is constituted by 9 ml of PBS+1 ml PBS containing 10,000 Legionella pneumophila.

Tube 2: Control resin: 9 ml of PBS+1 ml of PBS containing 10,000 Legionella pneumophila+0.1 g of P1S

Tube 3: 9 ml of PBS 4-1 ml of PBS containing 10,000 Legionella pneumophila+0.1 g of P3S

TABLE 18
quantification of the DNA of suspensions of
L. pneumophila placed in the presence of P3S

| | | Suspension extracted, purified + PCR | Relative |
|---|---|---|---|
| | | results in 10 ml of susp. | result |
| Tube 1 | Control | 1.06 · 10⁶ | 100 |
| | 10000/ml | | |
| Tube 2 | Control TR | 3.26 · 10⁵ | 31 |
| Tube 3 | P3S | 3.59 · 10⁴ | 3 |

A PCR control was carried out as follows. After 96 hours of contact the resins are allowed to settle at the bottom of the tubes, 5 ml of suspension is filtered on a 0.45 μm polycarbonate membrane to extract the cells. These are washed, lysed by thermal and chemical shocks. The DNAs are purified, amplified by PCR, and counted.

These results confirm:
the action of the grafted dendrimer P3 on polyisocyanate
a non negligible effect of the control (P1 S) even if it appears smaller than that of P3S.

### Example 21

### Antimicrobial Activity of the Fluorescent GLDs

The experiments described in Example 18, repeated using the GLD P2 of Example 11B, have shown that the antimicrobial activity of the fluorescent GLDs was identical to that of the non-fluorescent GLDs.

The fluorescent GLDs can therefore be used to study the mode of action of the GLDs. By mode of action is meant antimicrobial mechanisms, and also mechanisms for the transport of drugs, monitoring in the circulatory system, fluorescent marking of targets by GLDs carrying specific antibodies.

### Example 22

### Benefit of the GLDs for the Production of Antibodies and Their Characterization

Histamine immunoconjugate derivatives were prepared with two carriers: bovine serum albumin (BSA) and the generation 3 GLD (see formula hereafter) according to the method described by Vandenabeele-Trambouze, O. et al. [39].

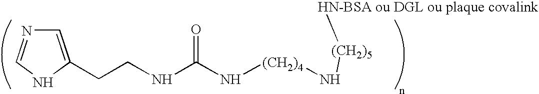

BSA ou DGL ou plaque covalink=BSA or DGL or covalink plate.

Structure of the immunoconjugate between histamine (representing a model hapten) and the carriers BSA, GLD and the Covalink plates.

The couplings are carried out with 5 mg of haptens, 20 mg of BSA or GLD and 300 μl of 5% glutaraldehyde in a 1.5 molar sodium acetate buffer, pH 8, and reduced to 3 N NaBH₄. 3 lots of two rabbits were immunized as follows:

Lot A: 150 μg of immunoconjugate-BSA in Freund's complete adjuvant at J=0, then 150 μg of conjugate-DGL in Freund's complete adjuvant at D=21, D=36 and D=51.

Lot B: 150 μg of conjugate-GLD in Freund's complete adjuvant at D=0, D=21, D=36 and D=51.

Lot C: 300 μg of native GLD in Freund's complete adjuvant at D=0, D=21, D=36 and D=51.

The sera of lots A, B, C, were analyzed at D=21, D=36, D=51 and D=65 according to two ELISA assays:
on a Maxisorp plate (NUNC) previously coated with immunoconjugates of BSA or of GLD (FIGS. 13 and 15) or the free carriers according to the method described by Vandenabeele-Trambouze, O. et al. [39]. This method makes it possible to assess the total antibodies titre (specific to the antigen or the hapten and specific to the carrier) as well as the rate of anti-carrier antibodies.
on a Covalink plate (NUNC) (FIG. 14) previously coated with hapten (according to the protocol described by Claeys-Bruno et al. [40, 41]). This method allows a direct assessment of the specific antibodies only.

The absence of measurable antibodies at D65 (number of days=65) for lots C and B (FIG. 13) demonstrated the immuno-stealth of the generation 3 GLD, including when the latter was grafted to a hapten. Similarly, the absence of ELISA response for LOT A and C at D65 on a Maxisorp plate coated with the native GLD (white circles and crosses—FIG. 13) confirms the absence of anti-GLD antibodies and therefore their immuno-stealth. This result is confirmed on Covalink plates for LOT B (FIG. 14, dashes) which does not show any anti-hapten antibodies.

However, when the GLD is used as a second carrier (LOT A) after stimulation of the immune system by an immunogenic carrier (BSA), it is noted, on the one hand, that an increase takes place in the titre of hapten-specific antibodies (see FIG. 14 between D21 and D36) and on the other hand the disappearance of the anti-BSA antibodies (carrier) after several immunizations with the GDL-hapten (FIG. 15). These results as a whole demonstrate that the GLDs, although immuno-stealth, make it possible to increase the titre of hapten-specific antibodies by simultaneously reducing the count of anti-carrier non-specific antibodies used to trigger the initial immune response.

### REFERENCES

[1]. Denkewalter, R. G., J. Kolc, and W. J. Lukasavage, Macromolecular highly branched homogeneous compound based on lysine units., in USP. 1981, Allied Corp.: US.
[2]. Denkewalter, R. G., J. Kolc, and W. J. Lukasavage, Macromolecular highly branched homogeneous compound, in USP. 1983, Allied Corp.: US.
[3]. Juan Rodriguez-Hernandez, Marco Gatti, and Harm-Anton Klok, Highly Branched Poly(L-lysine). Biomacromolecules, 2003. 4: p. 249-258.
[4]. Klok Harm-Anton and Rodriguez-Hernandez Juan, Dendritic-Graft Polypeptides. Macromolecules, 2002. 35: p. 8718-8723.
[5]. Matthews Barry Ross, et al., Agent for the prevention and treatment of sexually transmitted diseases-I., in World Intellectual Property Organization (PCT). 2002, Starpharma Limited: USA. p. 36.
[6]. Matthews Barry Ross and H. George, Anionic or cationic dendrimer antimicrobial or antiparasitic compositions., in USPTO. 2003, Starpharma Limited: USA.
[7]. Baigude H., et al., Synthesis of sphere-type monodispersed oligosaccharide-polypeptide dendrimers. Macromolecules, 2003. 36(19): p. 7100-7106.
[8]. Menz T. L. and Chapman T., Synthesis and characterization of hyperbranched polylysine. Polymer Preprints, 2003. 44(2): p. 842-843.
[9]. Takuro, N., et al., Method of gene delivery into cells using dendritic poly(L-lysine)s. peptide science, 2001. 2000 (37th): p. 201-204.
[10]. Choi Joon Sig, et al., Synthesis of a barbell-like triblock copolymer, poly(L-lysine) dendrimer-bloc-poly(ethylene glycol)-bloc-poly(L-lysine) dendrimer, and its self-assembly with plasmid DNA. Journal of American Chemical Society., 2000. 122: p. 474-480.
[11]. Kricheldorf, H. R., α-Aminoacid-N-Carboxy-Anhydrides and Related Heterocycles. Synthesis, Properties, Peptide Synthesis, Polymerisation. Heterocycles. 1987, Berlin: Springer-Verlag. 213 pages.
[12]. Bartlett, P. and R. Jones, A Kinetic Study of the Leuchs Anhydrides in Aqueous Solution. J. Am. Chem. Soc., 1957. 79(9): p. 2153-2159.
[13]. Commeyras, A., et al., Prebiotic Synthesis of Sequential Peptides on the Hadean Beach by a Molecular Engine Working with Nitrogen Oxides as Energy Sources. Polymer International., 2002. 51: p. 661-665.
[14]. Denkewalter, R. G. S., H.; Strachan, R. G.; Beesley, Thomas E.; Veber, Daniel F.; Schoenwaldt, Erwin F.; Barkemeyer, H.; Paleveda, William J., Jr.; Jacob, Theodore A.; Hirschmann, Ralph., Controlled synthesis of peptides in aqueous medium. I. Use of α-amino acid N-carboxyanhydrides. Journal of the American Chemical Society 1966. 88(13): p. 3163-4.
[15]. Hirschmann R., et al., The controlled Synthesis of peptides in aqueous medium. III. Use of Leuchs' anhydrides in the synthesis of dipeptides. Mécanism and control of side reactions. Journal of Organic Chemistry 1967. 32(11): p. 3415-3425.
[16]. Iwakura Y., et al., Stepwise synthesis of oligopeptides with N-carboxy α-amino acid anhydrides. Biopolymers, 1970. 9: p. 1419-1427.
[17]. Hitz T. H. and Luisi P. L., Spontaneous onset of homochirality in oligopeptide chain generated in the polymerisation of N-carboxyanhydride amino acids in water. Origin of life and evolution of the biosphere, 2004. 34: p. 93-110.
[18]. Sakamoto M. and Kuroyanagi Y., Multichain copoly(α-aminoacid). Journal of polymer science, 1978. 16: p. 1107-1122.
[19]. Birchall A. C. and North M., Synthesis of highly branched block copolymers of enantiomerically pure amino acids. Chem. Commun., 1998: p. 1335-1336.
[20]. Zigang Yang, Jianjun Yuan, and Shiyuan Cheng, Self-assembling of biocompatible BAB amphiphilic triblock copolymers PLL(Z)-PEG-PLL(Z) in aqueous medium. European polymer journal., 2005. 41: p. 267-274.
[21]. Poche D., Moore M., and Bowles J., An unconventional method for purifying the N-carboxyanhydride derivatives of γ-alkyl-L-glutamates. Synthetic communications, 1999. 29(5): p. 843-854.
[22]. Rekker R. F., The hydrophobic fragmental constant., ed. P. library. Vol. Vol. 1. 1977, Amsterdam: Elsevier.
[23]. Leuchs, H., Ber. Dtsch. Chem. Ges., 1906. 39: p. 857-859.
[24]. Curtius, T., et al., J. Prakt. Chem., 1930. 125: p. 211-302.
[25]. Coleman D. and Farthing A. C., J. Chem. Soc., 1951: p. 3218-3222.
[26]. Mobashery, S. and M. Johnston, A new approach to the preparation of N-carboxy α-amino acid anhydrides. J. Org. Chem., 1985. 50: p. 2200-2202.
[27]. Daly, W. H. and D. Poche, The preparation of N-carboxyanhydrides of α-amino acids using bis(trichloromethil)carbonate. Tetrahedron Lett., 1988. 29: p. 5859-5862.
[28]. Wilder, R. and S. Mobashery, The use of triphosgene in preparation of N-carboxy-α-amino acid anhydrides. J. Org. Chem., 1992. 57: p. 2755-2756.
[29]. Collet, H., et al., A new simple and quantitative synthesis of α-Aminoacid-N-Carboxyanhydrides. Tetrahedron Letters, 1996. 37(50): p. 9043-9046.
[30]. Pascal R., Boiteau L., and Commeyras A., From the prebiotic synthesis of α-aminoacids, towards a primitive translation apparatus for the synthesis of peptides. Top. Curr. Chem., 2005 (259): p. 69-122.
[31]. Fréchet, J. M. and D. A. Tomalia, eds. Dendrimers and other dendritic polymers., ed. W.s.i.p. science. 2001, John Wiley & Sons, Ltd. 646.
[32]. Commeyras, A., et al., Procédé de synthèse peptidique à partir des N—(N′-Nitroso)carbamoylaminoacides, in patent, PCT/FR 95/01380. 1995: Fr.
[33]. Matthews Barry Ross, et al., Agent for the prevention and treatment of sexually transmitted diseases-II., in World Intellectual Property Organization. 2002, Starpharma Limited: USA. p. 30.
[34]. Bello, M. S., R. Rezzonico, and P. G. Righetti, Use of Taylor-Aris dispersion for measurement of a solute diffusion coefficient in thin capillaries. Science, 1994. 266: p. 773.
[35]. Mes E. P. C., et al., Comparison of methods for the determination of diffusion coefficients of polymers in dilute solutions: the influence of polydispersity. Journal of Polymer Science: Part B: Polymer Physics, 1999. 37: p. 593-603.
[36]. Edward J. T., Molecular volumes and the stokes-einstein equation. Journal of Chemical Education, 1970. 47: p. 261.
[37]. Fehlbaum, P., et al., Insect immunity. Septic injury of Drosophila induces the synthesis of a potent antifungal peptide with sequence homology to plant antifungal peptides. J. Biol. Chem., 1994. 269: p. 33159-33163.
[38]. Hancock, R. E., T. Falla, and M. Brown, Cationic bactericidal peptides. Adv. Microb. Physiol., 1995. 37: p. 135-175.
[39]. Vandenabeele-Trambouze, O., et al., Antibodies directed against L and D isovaline using a chemical derivatizating reagent for the measurement of their enantiomeric ratio in extraterrestrial samples: first step-production and characterization of antibodies. Chirality, 2002. 14: p. 519-526.
[40]. Claeys-Bruno, M., et al., Methodological approaches for histamine quantification using derivatization by chloroethylnitrosourea and ELISA measurement. Part I: Optimisation of derivated histamine detection with coated-plates using optimal design. Chemometrics and intelligent Laboratory Systems, 2006. 80, ((2)): p. 176-185.
[41]. Claeys-Bruno, M., et al., Methodological approaches for histamine quantification using derivatization by chloroethylnitrosourea and ELISA measurement. Part II: Optimisation of the derivatization step. Chemometrics and intelligent Laboratory Systems, 2006. 80((2),): p. 186-197.

1-30. (canceled)

31. Method for the preparation of hydrophobic polypeptides in the form of precipitates by polymerization of activated a-amino acid monomers in an aqueous solvent, said polypeptide being capable of being resolubilized in said solvent.

32. Method according to claim 31, in which the activated a-amino acids are chosen from a-amino acid N-carboxyanhydrides (NCA), a-amino acid N,N′carbonyldiimidazoles, a-amino acid carbonyl sulphide, a-amino acid carbonic anhydride, and amino thioacids-oxidizing agents.

33. Method according to claim 31, of a-amino acid N-carboxyanhydrides (NCA) of the following formula (1): 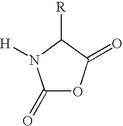
in which R represents a side chain of a natural or modified a-amino acid.

34. Method according to claim 32, of L-lysine-NCA monomers for the preparation of polylysine in aqueous solvent.

35. Method for the preparation of a grafted homo or heteropolylysine dendrimer, from a primer comprising at least one primary or secondary amine group, comprising a step of addition of an L-lysine-NCA monomer, and optionally one or more other a-amino-acid-NCA monomers to said primer in an aqueous solvent.

36. Method for the preparation of a grafted homo or heteropolylysine dendrimer according to claim 35, wherein the a-amino-acid-NCA monomers are chosen from the list comprising L-ornithine-NCA, L-glutamic-acid-NCA and its y-amide, L-aspartic-acid-NCA and its β-amide, L-diamino-2,4,-butyric-acid-NCA and its β-amide, L-tyrosine-NCA, L-serine-NCA, L-threonine-NCA, L-phenylalanine-NCA, L-valine-NCA, L-leucine-NCA, L-isoleucine-NCA, L-alanine-NCA, and glycine-NCA.

37. Method for the preparation of a grafted homo or heteropolylysine dendrimer according to claim 35, in which the monomer is L-lysine-NCA only.

38. Method for the preparation of a grafted homo or heteropolylysine dendrimer according to claim 35, in which the L-lysine-NCA is NE-protected, by a group.

39. Method for the preparation of a grafted homo or heteropolylysine dendrimer according to claim 38, wherein the group is chosen from:
—COH (Formyl), —COCF₃ (TFA), —OCOC(CH₃)₃ (Boc), —COOCH₂Φ (Z),

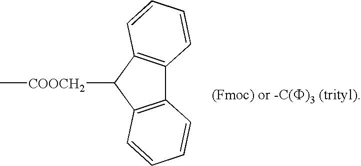

40. Method for the preparation of a grafted homo or heteropolylysine dendrimer according to claim 37, in which the primer is chosen from L-lysine, L-ornithine, a homopolylysine, a poly(ethylene glycol)-a,ω-diamine, a heteropolylysine, a heteropeptide, and a homopeptide.

41. Method for the preparation of a grafted homo or heteropolylysine dendrimer according to claim 37, in which the pH of the solvent is 3 to 9.

42. Method for the preparation of a grafted homo or heteropolylysine dendrimer according to claim 37, said polylysine dendrimer being of generation 1, comprising the following steps:
addition of the N^{ε}-protected L-lysine-NCA to a primer in an aqueous solvent, at an appropriate pH, in order to obtain a protected polylysine dendrimer of generation 1 in the form of a precipitate,
deprotection of the protected polylysine dendrimer of generation 1 obtained in the previous step, in order to obtain the polylysine dendrimer of generation 1.

43. Method for the preparation of a grafted homopolylysine dendrimer according to claim 42, said polylysine dendrimer being of generation 1, in which the primer is N^{ε} protected or unprotected L-lysine.

44. Method for the preparation of a grafted heteropolylysine dendrimer according to claim 37, said polylysine dendrimer being of generation 1, in which the primer is a poly(ethylene glycol)-a,ω-diamine, the molecular weight of which is 100 Da to 10,000 Da, preferably 1,000 Da to 10,000 Da.

45. Method for the preparation of a grafted homopolylysine dendrimer according to claim 42, said polylysine dendrirner being of generation 1, comprising:
a step of addition of L-lysine-NCA N^{ε}-protected by a TFA group, to an aqueous solution with a pH of 6 to 8, without addition of primer or with an L-lysine primer N^{ε}-protected by a TFA group, in order to obtain a precipitate of protected polylysine dendrimer of generation 1, and
a step of deprotection of the polylysine polymer obtained in the previous step in order to obtain a linear polylysine dendrimer of generation 1, of molecular weight 1,450 Da, having a polydispersity index of 1.2 and corresponding to an average degree of polymerization of 8 units of lysine.

46. Method for the preparation of a grafted homo or heteropolylysine dendrimer according to claim 37, in which the grafted polylysine dendrimer is of generation n, n being an integer from 2 to 10, comprising:
a step of addition of N^{ε}-protected L-lysine-NCA to a primer constituted by a grafted polylysine dendrimer of generation n−1, in an aqueous solvent, at an appropriate pH, in order to obtain the protected grafted polylysine dendrimer of generation n in the form of a precipitate,
said grafted polylysine dendrimer of generation n−1 being itself obtained from the addition of N′-protected L-lysine-NCA to a primer constituted by a grafted polylysine dendrimer of generation n−2, in an aqueous solvent, at an appropriate pH, in order to obtain a protected grafted polylysine dendrimer of generation n−1, and the deprotection of said polylysine,
said grafted polylysine dendrimer of generation n−2 being itself obtained as indicated in relation to the grafted polylysine dendrimer of generation n−1,
and when n=2, the polylysine dendrimer of generation 1 is as previously defined, said polylysine dendrimer of generation 1 forming the core of the polylysine dendrimer of generation n,
a step of deprotection of the protected grafted polylysine dendrimer of generation n to obtain the grafted polylysine dendrimer of generation n.

47. Method for the preparation of a grafted homopolylysine dendrimer according to claim 46, said grafted polylysine dendrimer being of generation n, in which the L-lysine-NCA is N^{ε}-protected by TFA, the core of said grafted homopolylysine dendrimer of generation n being formed from a linear polylysine comprising 8 residues of L-lysine, and the degree of branching of said grafted homopolylysine dendrimer of generation n being 40% to 100%.

48. Method for the preparation of a grafted homopolylysine dendrimer according to claim 46, said grafted polylysine dendrimer being of generation 2, comprising:
a step of addition of the L-lysine-NCA N^{ε}-protected by TFA to a primer constituted by a polylysine dendrimer of generation 1,
the mass ratio (L-lysine-NCA N^{ε}-protected by TFA)/(polylysine dendrimer of generation 1) being 2.6 to 3.9,
said step of addition taking place in an aqueous solvent, at an appropriate pH, in order to obtain a protected polylysine dendrimer of generation 2,
a step of deprotection of the polylysine obtained in the previous step, in order to obtain a polylysine dendrimer of generation 2 having an average molecular weight of 6,000 to 14,000 Da, a polydispersity of 1.4, and 40 to 60, free external —NH₂ groups.

49. Method for the preparation of a grafted homopolylysine dendrimer according to claim 48, wherein the polylysine dendrimer of generation 2 has an average molecular weight of 8,350 Da.

50. Method for the preparation of a grafted homopolylysine dendrimer according to claim 48, wherein the polylysine dendrimer of generation 2 has an average molecular weight of 8,600 Da.

51. Method for the preparation of a grafted homopolylysine dendrimer according to claim 46, said grafted polylysine dendrimer being of generation 3, comprising:
a step of addition of the L-lysine-NCA N^{ε}-protected by TFA to a primer constituted by a generation 2 grafted polylysine dendrimer,
the mass ratio (L-lysine-NCA NE-protected by TFA)/(generation 2 grafted polylysine dendrimer) being 2.6 to 3.9,
said step of addition taking place in an aqueous solvent, at an appropriate pH, in order to obtain a protected grafted polylysine dendrimer of generation 3,
a step of deprotection of the polylysine obtained in the previous step, in order to obtain a polylysine dendrimer of generation 3 having an average molecular weight of 15,000 to 30,000 Da, a polydispersity of 1.4, and 100 to 150 free external —NH₂ groups.

52. Method for the preparation of a grafted homopolylysine dendrimer according to claim 51, wherein the polylysine dendrimer of generation 3 has an average molecular weight of 21,500 Da.

53. Method for the preparation of a grafted homopolylysine dendrimer according to claim 51, wherein the polylysine dendrimer of generation 3 has an average molecular weight of 22,000 Da.

54. Method for the preparation of a grafted homopolylysine dendrimer according to claim 46, said grafted polylysine dendrimer being of generation 4, comprising:
a step of addition of the L-lysine-NCA N^{ε}-protected by TFA to a primer constituted by a generation 3 grafted polylysine dendrimer,
the ratio (L-lysine-NCA N^{ε} protected by TFA)/(generation 3 grafted polylysine dendrimer) being 2.6 to 3.9,
said step of addition taking place in an aqueous solvent, at an appropriate pH, in order to obtain a protected grafted polylysine dendrimer of generation 4,
a step of deprotection of the polylysine obtained in the previous step, in order to obtain a grafted polylysine dendrimer of generation 4 having an average molecular weight of 50,000 to 80,000 Da, a polydispersity of 1.4, and 300 to 450 free external —NH₂ groups.

55. Method for the preparation of a grafted homopolylysine dendrimer according to claim 54, wherein the grafted polylysine dendrimer of generation 4 has an average molecular weight of 64,000 Da.

56. Method for the preparation of a grafted homopolylysine dendrimer according to claim 54, wherein the grafted polylysine dendrimer of generation 4 has an average molecular weight of 65,300 Da.

57. Method for the preparation of a grafted homopolylysine dendrimer according to claim 46, said grafted polylysine dendrimer being of generation 5, comprising:
a step of addition of the L-lysine-NCA N^{ε}-protected by TFA to a primer constituted by a generation 4 grafted polylysine dendrimer,
said generation 4 grafted polylysine dendrimer having an average molecular weight of 50,000 to 80,000 Da, a polydispersity of 1.4, and 300 to 450 free external —NH₂ groups,
the ratio (L-lysine-NCA N^{ε}-protected by TPA)/(generation 4 grafted polylysine dendrimer) being 2.6 to 3.9,
said step of addition taking place in an aqueous solvent, at an appropriate pH, in order to obtain a protected polylysine dendrimer of generation 5,
a step of deprotection of the polylysine obtained in the previous step, in order to obtain a grafted polylysine dendrimer of generation 5 having an average molecular weight of 140,000 to 200,000 Da, a polydispersity of 1.5, and 900 to 1,100 free external —NH₂ groups.

58. Method for the preparation of a grafted homopolylysine dendrimer according to claim 57, wherein the grafted polylysine dendrimer of generation 5 has an average molecular weight of 169,000 Da.

59. Method for the preparation of a grafted homopolylysine dendrimer according to claim 57, wherein the grafted polylysine dendrimer of generation 5 has an average molecular weight of 172,300 Da.

60. Method for the preparation of a grafted homo- or hetero polylysine dendrimer according to claim 35, in which the primer is fixed covalently to the grafted dendrimer, said primer comprising a detectable marker product.

61. Grafted polylysine dendrimer such as can be obtained by the implementation of a method according to claim 37.

62. Grafted polylysine dendrimer according to claim 61, characterized in that the external —NH₂ groups are bonded covalently or non-covalently to groups chosen from the list comprising monosaccharides, nucleic acids, proteins, groups having carboxylic, sulphonic and phosphoric functions, ethylene polyoxides and hydrocarbonated or perfluorohydrocarbonated chains, aldehydes and their precursor, carbamoyl and chloroethylnitrosourea groups.

63. Grafted polylysine dendrimer according to claim 61, characterized in that it is fixed covalently or non-covalently onto a support.

64. Grafted polylysine dendrimer according to claim 63, characterized in that it is fixed covalently or non-covalently onto a support by electrostatic bonds.

65. Grafted polylysine dendrimer according to claim 61, characterized in that it is furtive vis-ã-vis the immune systems with which it is brought into contact, and in that it is advantageously used as a carrier of haptens or antigens against which the immune systems react to form antibodies.

66. Method for the preparation of antigen-grafted polylysine dendrimer complexes or hapten-grafted polylysine dendrimer complexes by use of a grafted polylysine dendrimer according to claim 61, said complexes being intended for the production of antibodies directed against said antigen or said hapten.

67. Composition of grafted polylysine dendrimers such as can be obtained by the implementation of a method according to claim 37.

68. Grafted polylysine dendrimer according to claim 61, as an antibacterial or antifungal, with the proviso that it is not used for the therapeutic treatment of the human or animal body.

69. Pharmaceutical composition, characterized in that it comprises, as an active ingredient at least one grafted polylysine dendrimer according to claim 61, in combination with a pharmaceutically acceptable vehicle.

70. Method for the preparation of a medicament intended for the treatment of bacterial or fungal infections, or cancers by use of a grafted polylysine dendrimer according to claim 61.

71. Method according to claim 70, in which the bacterial infections are chosen from infections by GRAM− and GRAM+ bacteria belonging to the families chosen from the list comprising Pseudoinonadaceac, Legionellaceae, Enterobacteriaceae, Vibrionaceae, Pasteurellaceac, Alcaligenaceae, Brucellaceae, Francisellaceae, Neisseriaceae, Micrococcaceae.
